(19) United States
(12) Patent Application Publication
(10) Pub. No.: US 2024/0082435 A1
(43) Pub. Date: Mar. 14, 2024

(54) Solid phase synthesis of glutamate-urea-lysine derived (GUL derived) prostate-specific membrane antigen (PSMA) targeting conjugates and their use as precursors for therapeutic and/or diagnostic agents

(71) Applicant: TELIX INTERNATIONAL (INNOVATIONS) PTY LTD, Victoria (AU)

(72) Inventors: Artur Pinto, Roissy CDG Cedex (FR); Alexis Bontemps, Roissy CDG Cedex (FR); Lucas Lemaire, Roissy CDG Cedex (FR)

(21) Appl. No.: 18/547,286

(22) PCT Filed: Feb. 25, 2022
(86) PCT No.: PCT/AU2022/050154

§ 371 Date: Aug. 21, 2023

(30) Foreign Application Priority Data
Feb. 26, 2021 (AU) 2021900532
Oct. 26, 2021 (AU) 2021903428

(51) Int. Cl. A61K 51/04

(52) U.S. Cl. CPC A61K 51/0482; A61K 51/0455; A61K 2121/00; A61K 2123/00

(57) ABSTRACT

The disclosure provides methods for the solid phase synthesis of glutamate-urea-lysine derived (GUL derived) prostate-specific membrane antigen (PSMA) targeting conjugates. The disclosure also relates to key intermediates of this process and methods for their preparation such as a method of preparing a compound of formula (V). Also disclosed are use of the conjugates as precursors for therapeutic and/or diagnostic agents, including treating and diagnosing prostate cancer.

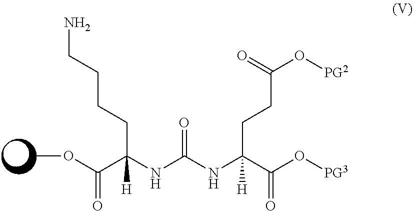

### RELATED APPLICATIONS

This application claims priority from Australian provisional application nos. 2021903428 (filed on 26 Oct. 2021) and 2021900532 (filed on 26 Feb. 2021). The entire contents of each of AU 2021903428 and AU 2021900532 are incorporated herein by reference.

### FIELD OF THE INVENTION

The invention relates to a solid phase synthesis of a prostate-specific membrane antigen (PSMA)-binding moiety.

### BACKGROUND OF THE INVENTION

Glutamate-urea-lysine (GUL; compound 1) can serve as a binding moiety for PSMA.

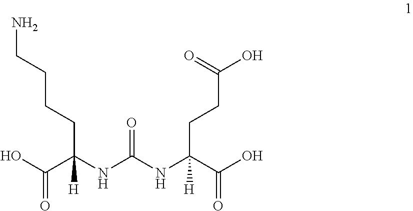 [SMILES: NCCCC[C@H](NC(=O)N[C@@H](CCC(=O)O)C(=O)O)C(=O)O]

The structure and solution phase synthesis of compound 1 has been described in U.S. Pat. Nos. 9,309,193, 9,878,980, 10,640,461 and J. Med. Chem. 44:298;2001.

PSMA is a target of interest in treating various forms of prostate cancer, including castration-resistant prostate cancer (CRPC). One approach for targeting PSMA in prostate cancer therapy is targeted radionuclide therapy, for example by treatment with a radionuclide-bearing ligand linked to a PSMA-binding moiety.

Targeting PSMA may also be useful in prostate cancer diagnosis/prognosis as linking a PSMA-targeting moiety with an imaging agent can be used in a variety of techniques sensitive to radionuclide concentrations (eg. positron emission tomography (PET), single-photon emission computerized tomography (SPECT), etc.) to obtain images of a prostate cancer tumour. This targeted imaging may also be useful to monitor the effectiveness of prostate cancer therapy by tracking tumour size over time.

Accordingly, there exists a continuing need to provide alternative syntheses of GUL.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

The invention provides a solid phase synthesis of GUL. Solid phase synthesis avoids the need for many separation steps associated with solution phase syntheses. The solid phase synthesis of the invention also provides a flexible approach where various radionuclide ligands can be linked with GUL, providing a platform for forming a conjugate of GUL with a desired radionuclide ligand.

In one aspect, there is provided a method of preparing a compound of formula (V) or a salt thereof

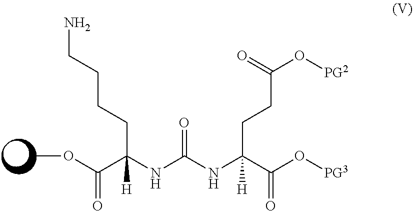

wherein
[symbol] denotes a solid support; and
PG² and PG³ are independently carboxyl protecting groups,
the method comprising reacting a compound of formula (II) with a compound of formula (III) followed by selective deprotection of PG¹,

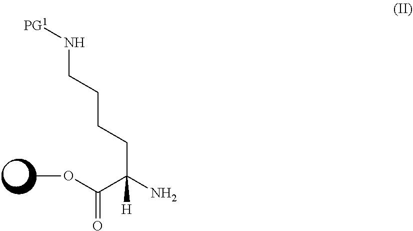

wherein:
[symbol] denotes a solid support; and
PG¹ is an amine protecting group

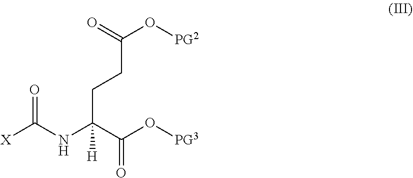

wherein:
X is a carbonyl activating group; and
PG² and PG³ are as defined for formula (V).

In another aspect, there is provided a method of preparing a compound of formula (IV) or a salt thereof

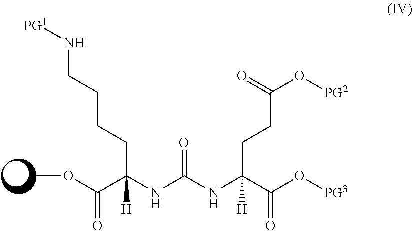

wherein
[symbol] denotes a solid support;
PG¹ is an amine protecting group; and
PG² and PG³ are independently carboxyl protecting groups;
the method comprising reacting a compound of formula (II) with a compound of formula (III),

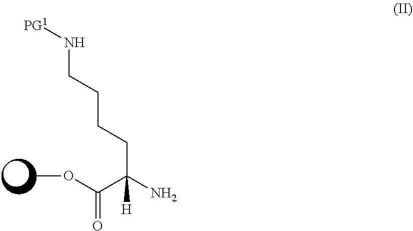

wherein:
[symbol] denotes a solid support; and
PG¹ is an amine protecting group

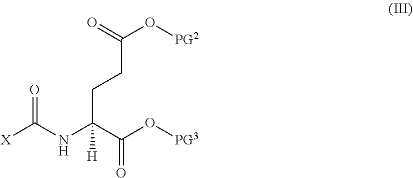

wherein:
X is a carbonyl activating group; and
PG² and PG³ are as defined for formula (IV).

In another aspect, there is provided a method of preparing a compound of formula (I) or a salt thereof:

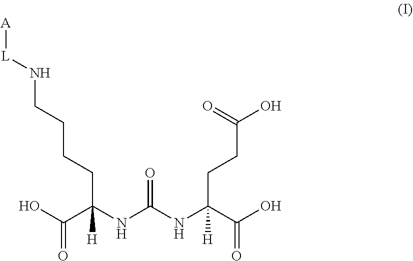

wherein
L is a covalent bond or a bifunctional linker; and
A is a ligand for a diagnostic or therapeutic agent;
the method comprising:
   reacting the compound of formula (V) as defined herein with a compound of formula (VI), formula (VII) or formula (VIII) followed by deprotection (typically comprising cleaving PG² and PG³ simultaneously or in separate steps) and cleavage from the solid support to provide the compound of formula (I)

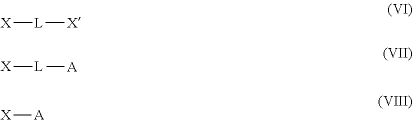

wherein
L is as defined for formula (I), with the proviso that in formula (VI), L is a bifunctional linker;
A is as defined for formula (I)
X is a group cleavable in the reaction with the compound of formula (V)
X′ is H or a group cleavable in a subsequent step reacting moiety L with a synthon of moiety A.

In another aspect, also provided is a method of preparing a compound of formula (I), the method comprising cleaving a compound of formula (X)

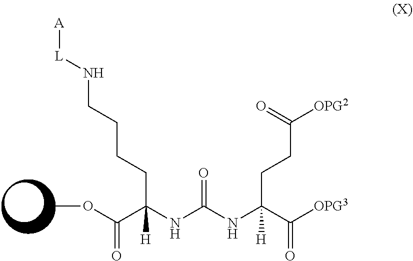

wherein
[symbol] denotes a solid support; and
L is a covalent bond or a bifunctional linker or a protected form thereof;
A is a ligand for a diagnostic and/or therapeutic agent or a protected form thereof; and
PG² and PG³ are independently H or a carboxyl protecting group,
from the solid support, and
optionally deprotecting the compound (typically comprising cleaving PG² and PG³ simultaneously during the cleaving step or in one or more separate steps before or after the cleaving step).

In another aspect, there is provided a method of preparing a compound of formula (I), the method comprising
reacting a compound of formula (IX)

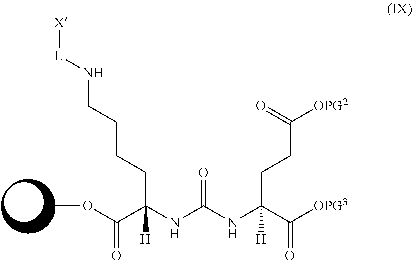

wherein
L is a bifunctional linker;
X′ is H or a group cleavable in a subsequent step reacting moiety L with a synthon of moiety A; and
PG² and PG³ are independently H or a carboxyl protecting group,
with LG_{L}-A
wherein
LG_{L} is H or a group cleavable to form a bond from moiety L to moiety A; and
A is a ligand for a therapeutic or diagnostic agent or a protected form thereof, to provide a compound of formula (X)

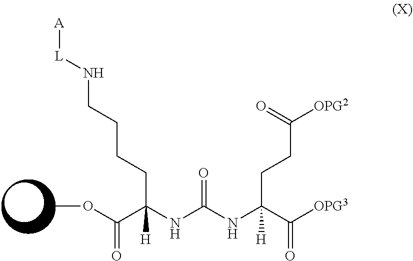

wherein
[symbol] denotes a solid support; and
L is a bifunctional linker;
A is a ligand for a diagnostic and/or therapeutic agent or a protected form thereof; and
PG2 and PG3 are independently H or a carboxyl protecting group,
cleaving the compound of formula (X) from the solid support, and
optionally deprotecting the compound (typically comprising cleaving PG2 and PG3 simultaneously during the cleaving step or in one or more separate steps before or after the cleaving step).

In another aspect, there is provided a method of preparing a compound of formula (XII) or a salt thereof

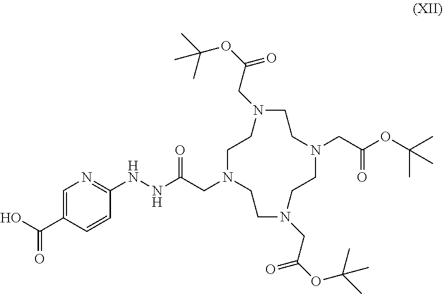 [SMILES: CC(C)(C)OC(=O)CN1CCN(CC(=O)NNc2ccc(C(=O)O)cn2)CCN(CC(=O)OC(C)(C)C)CCN(CC(=O)OC(C)(C)C)CC1]

the method comprising deprotecting PG⁴ of a compound of formula (XIII)

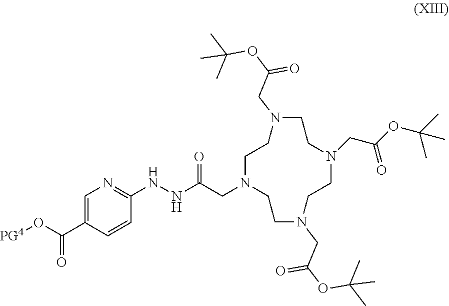

wherein
PG⁴ is a carboxyl protecting group orthogonal with a tert-butyl ester protecting group.

In another aspect, there is provided a method of preparing a compound of formula (V) comprising reacting a compound of formula (II) with a compound of formula (III) followed by optional deprotection. The compounds of formulas (V), (II) and (III) may be as defined in any aspect or embodiment described herein.

In another aspect, there is provided a method of preparing a compound of formula (V) or a salt thereof, the method comprising selective deprotection of PG¹ in the compound of formula (IV) to provide the compound of formula (V). The compounds of formulas (V) and (IV) may be as defined in any aspect or embodiment described herein.

In another aspect there is provided a compound of formula (IV), the method comprising reacting a compound of formula (II) and a compound of formula (III).

In another aspect, there is provided a compound prepared by or obtainable from the methods of the invention. In some embodiments, the compound is a compound of formula (I) prepared by the methods of the invention. In some embodiments, the compound is a compound (alternatively an intermediate compound) of formula (XII) prepared by methods of the invention.

In another aspect, there is provided a compound of any one of formulas (II)-(X) (including compounds of formula (II), (Ill), (IV), (V), (V′), (VI), (VII), (VIII), (IX) and (X)) or a salt thereof.

In another aspect, there is provided a compound (alternatively an intermediate compound) of any one of formulas (XIII), (XIV) and (XVI) or a salt thereof.

In a further aspect, there is provided a pharmaceutical composition comprising a compound of the invention optionally complexed with a therapeutic and/or diagnostic agent, such as a radio isotope.

In yet another aspect, there is provided a method of treating prostate cancer, comprising administering to a subject in need thereof an effective amount of a complex of a therapeutic radio isotope and a compound of formula (I) of the invention or a pharmaceutically acceptable salt thereof, or a composition comprising the complex.

In another aspect there is provided use of a compound of formula (I) of the invention or a pharmaceutically acceptable salt thereof in the preparation of a medicament for treating prostate cancer.

In another aspect, there is provided a method of imaging a prostate cancer tumour, comprising administering to a subject in need thereof an effective amount of a complex of a diagnostic radio isotope and a compound of formula (I) of the invention or a pharmaceutically acceptable salt thereof, or a composition comprising the complex, and imaging the prostate cancer tumour.

In a further aspect, there is provided a method of diagnosing, monitoring or prognosing a prostate cancer, comprising:
(a) administering to a subject in need thereof an effective amount of a complex of a diagnostic radio isotope and a compound of formula (I) of the invention or a pharmaceutically acceptable salt thereof, or a composition comprising the complex,
(b) allowing the complex to concentrate at sites in the subject where PSMA is found; and
(c) detecting the radio isotope of the complex
whereby detection of the radio isotope enables an image of the cancer (if present) to be created to thereby diagnose, monitor or prognose the prostate cancer.

### Selected Definitions

The term “alkyl” is intended to include saturated straight chain and branched chain hydrocarbon groups. In some embodiments, alkyl groups have from 1 to 12, 1 to 10, 1 to 8, 1 to 6, or from 1 to 4 carbon atoms. In some embodiments, alkyl groups have from 5-21, from 9-21, or from 11-21 carbon atoms, such as from 11, 13, 15, 17, or 19 carbon atoms. Examples of straight chain alkyl groups include, but are not limited to, methyl, ethyl, n-propyl, n-butyl, n-pentyl, n-hexyl, n-heptyl, and n-octyl. Examples of branched alkyl groups include, but are not limited to, isopropyl, iso-butyl, sec-butyl, tert-butyl, neopentyl, isopentyl, and 2,2-dimethylpropyl.

The term “ester” refers to a carboxylic acid group where the hydrogen of the hydroxyl group has been replaced by a saturated, straight-chain (i.e. linear) or branched hydrocarbon group. Specific examples of alkyl groups are methyl, ethyl, propyl, iso-propyl, n-butyl, iso-butyl, sec-butyl, ted-butyl, n-pentyl, iso-pentyl, n-hexyl and 2,2-dimethylbutyl. The alkyl group may be a C₁-C₆ alkyl group. As used herein a wording defining the limits of a range of length such as, for example, “from 1 to 5” means any integer from 1 to 5, i.e. 1, 2, 3, 4 and 5. In other words, any range defined by two integers explicitly mentioned is meant to comprise and disclose any integer defining said limits and any integer comprised in said range. The alkyl group may be a branched alkyl group.

The term “carboxyl protecting group” as used herein is intended to mean a group that is capable of being readily removed to provide the OH group of a carboxyl group and protects the carboxyl group against undesirable reaction during synthetic procedures. Such protecting groups are described in Protective Groups in Organic Synthesis edited by T. W. Greene et al. (John Wiley & Sons, 1999) and ‘Amino Acid-Protecting Groups’ by Fernando Albericio (with Albert Isidro-Llobet and Mercedes Alvarez) Chemical Reviews 2009 (109) 2455-2504. Examples include, but are not limited to, alkyl and silyl groups, for example methyl, ethyl, tert-butyl, methoxymethyl, 2,2,2-trichloroethyl, benzyl, diphenylmethyl, trimethylsilyl, and tert-butyldimethylsilyl, and the like.

The term “amine protecting group” as used herein is intended to mean a group that is capable of being readily removed to provide the NH₂ group of an amine group and protects the amine group against undesirable reaction during synthetic procedures. Such protecting groups are described in Protective Groups in Organic Synthesis edited by T. W. Greene et al. (John Wiley & Sons, 1999) and ‘Amino Acid-Protecting Groups’ by Fernando Albericio (with Albert Isidro-Llobet and Mercedes Alvarez) Chemical Reviews 2009 (109) 2455-2504. Examples include, but are not limited to, acyl and acyloxy groups, for example acetyl, chloroacetyl, trichloroacetyl, o-nitrophenylacetyl, o-nitrophenoxy-acetyl, trifluoroacetyl, acetoacetyl, 4-chlorobutyryl, isobutyryl, picolinoyl, aminocaproyl, benzoyl, methoxy-carbonyl, 9-fluorenylmethoxycarbonyl (fmoc), 2,2,2-trifluoroethoxycarbonyl, 2-trimethylsilylethoxy-carbonyl, tert-butyloxycarbonyl (BOC), allyloxycarbonyl (alloc), benzyloxycarbonyl, p-nitrobenzyloxycarbonyl, 2,4-dichloro-benzyloxycarbonyl, and the like. Further examples include Cbz (carboxybenzyl), Nosyl (o- or p-nitrophenylsulfonyl), Bpoc (2-(4-biphenyl)isopropoxycarbonyl) and Dde (1-(4,4-dimethyl-2,6-dioxohexylidene)ethyl).

The term “carboxamide protecting group” as used herein is intended to mean a group that is capable of being readily removed to provide the NH₂ group of a carboxamide group and protects the carboxamide group against undesirable reaction during synthetic procedures. Such protecting groups are described in Protective Groups in Organic Synthesis edited by T. W. Greene et al. (John Wiley & Sons, 1999) and ‘Amino Acid-Protecting Groups’ by Fernando Albericio (with Albert Isidro-Llobet and Mercedes Alvarez) Chemical Reviews 2009 (109) 2455-2504. Examples include, but are not limited to, 9-xanthenyl (Xan), trityl (Trt), methyltrityl (Mtt), cyclopropyldimethylcarbinyl (Cpd), and dimethylcyclopropylmethyl (Dmcp).

As used herein, the term ‘theranostic’ refers to the ability of compounds/materials to be used for diagnosis as well as for therapy. The term “theranostic reagent” relates to any reagent which is both suitable for detection, diagnostic and/or the treatment of a disease or condition of a patient. The aim of theranostic compounds/materials is to overcome undesirable differences in biodistribution and selectivity, which can exist between distinct diagnostic and therapeutic agents.

As used herein, the term “and/or” means “and”, or “or”, or both.

The term “(s)” following a noun contemplates the singular and plural form, or both.

As used herein, except where the context requires otherwise, the term “comprise” and variations of the term, such as “comprising”, “comprises” and “comprised”, are not intended to exclude further additives, components, integers or steps.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9, and 10) and also any range of rational numbers within that range (for example, 2 to 8, 1.5 to 5.5, and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

Various features of the invention are described with reference to a certain value, or range of values. These values are intended to relate to the results of the various appropriate measurement techniques, and therefore should be interpreted as including a margin of error inherent in any particular measurement technique. Some of the values referred to herein are denoted by the term “about” to at least in part account for this variability. The term “about”, when used to describe a value, may mean an amount within±10%, ±5%, ±1% or ±0.1% of that value.

Further aspects of the present invention and further embodiments of the aspects described in the preceding paragraphs will become apparent from the following description, given by way of example and with reference to the accompanying drawings.

### DETAILED DESCRIPTION OF THE EMBODIMENTS

The invention relates to methods of preparing a compound of formula (I) or a salt thereof

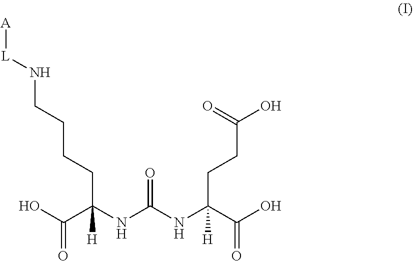

wherein
L is a covalent bond or a bifunctional linker; and
A is a ligand for a diagnostic or therapeutic agent.

The compounds of formula (I) link a GUL-derived PSMA-targeting moiety with a ligand for a diagnostic and/or therapeutic agent (moiety A). Accordingly, the compounds of formula (I) may be referred to as precursors of PSMA-targeted diagnostic and/or therapeutic agents.

Advantageously, the methods of the invention allow for the facile solid phase synthesis of compounds of formula (I). These methods may avoid the need for one or more purification/separation steps associated with the solution phase synthesis of similar compounds. These methods also provide useful synthetic intermediates serving as a platform for preparing various PSMA-targeting precursor therapeutic or diagnostic agents.

The method for preparing the compound of formula (I) or a salt thereof, comprises the steps of:
reacting a compound of formula (II) with a compound of formula (III) to provide a compound of formula (IV):

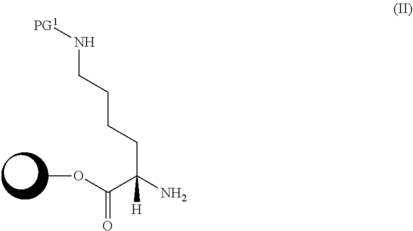

wherein:
[symbol] denotes a solid support; and
PG¹ is an amine protecting group

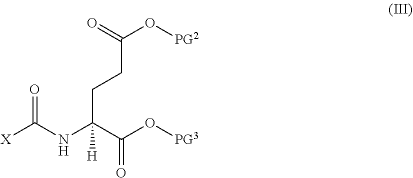

wherein:
X is a carbonyl activating group; and
PG² and PG³ are independently carboxyl protecting groups;

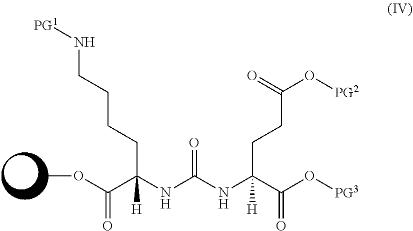

wherein PG¹-, PG² and PG³ are as defined for formulas (II) and (III)
   selectively deprotecting the compound of formula (IV) (eg comprising cleaving PG¹) to provide a compound of formula (V)

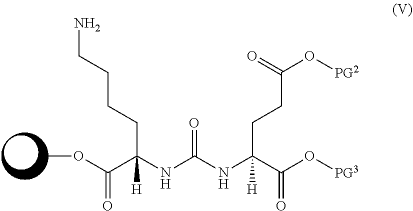

wherein PG² and PG³ are as defined for formula (III)
   reacting the compound of formula (V) with a compound of formula (VI), formula (VII) or formula (VIII) followed by deprotection (typically comprising cleaving PG² and PG³ simultaneously or in separate steps) and cleavage from the solid support to provide the compound of formula (I)

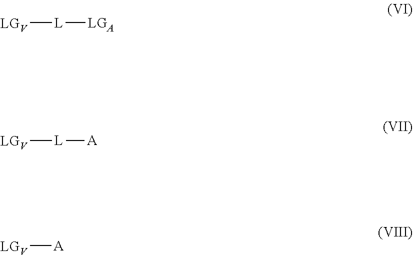

wherein
L is a bifunctional linker or a protected form of a bifunctional linker;
A is a ligand for a therapeutic and/or diagnostic agent or a protected form thereof;
LG_{V} is a group cleavable in the reaction with the compound of formula (V)
LG_{A} is H or a group cleavable in a subsequent step reacting moiety L with a synthon of moiety A.

### Compounds of Formula (I)

Provided herein are compounds of formula (I) prepared by the methods described herein.

### L

In some embodiments, L is a covalent bond. In these embodiments, the nitrogen atom of the lysine residue side chain is bonded directly to moiety A.

In some embodiments, L is a bifunctional linker. The bifunctional linker may be any diradical species capable of covalently linking the PUG moiety and the ligand together without interfering with the PSMA targeting function of PUG or the radionuclide complexation of the ligand.

Suitable bifunctional linkers include bromoacetyl, thiols, succinimide ester, tetrafluorophenyl (TFP) ester, a maleimide, amino acids (including natural and non-natural amino acids), a nicotinamide, a nicotinamide derivative, or using any amine or thiol- modifying chemistry known in the art.

In some embodiments, the bifunctional linker is selected from one or more amino acids, a nicotinamide and a nicotinamide derivative.

In some embodiments when the bifunctional linker is selected from one or more amino acids, the bifunctional linker may be a single amino acid, or two or more amino acids linked in a peptide chain. The peptide chain may comprise at least 2, 3, 4, 5, 6, 7, 8, 9, 10 or more amino acid residues. In some embodiments, the peptide chain may comprise a number of amino acid residues between any 2 of these values, for example from 2-10 residues or from 2-6 residues.

In some embodiments, the bifunctional linker is selected from:

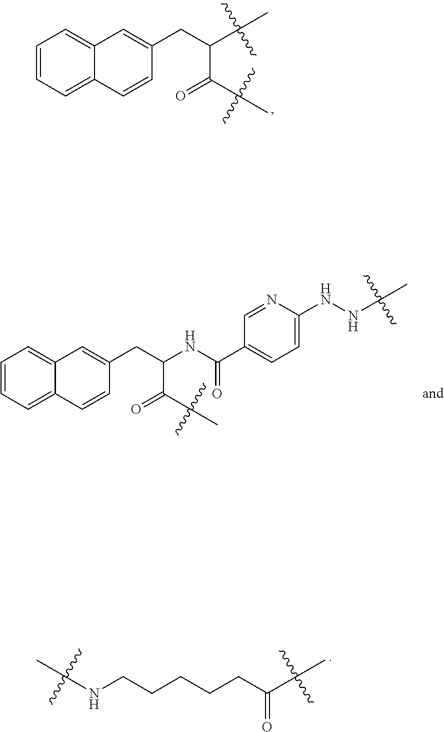 [SMILES: *C(=O)C(*)Cc1ccc2ccccc2c1 ; *NNc1ccc(C(=O)NC(Cc2ccc3ccccc3c2)C(*)=O)cn1 ; *NCCCCCC(*)=O]

In some embodiments, the bifunctional linker may comprise a chelating moiety. In these embodiments, the chelating moiety of the linker may bind a different radio nuclide(s) to the ligand. In some embodiments, the chelating moiety may be a 6-hydrazinylnicotinamide moiety, which may optionally be bound to a further ligand, for example DOTA. The 6-hydrazinylnicotinamide moiety typically binds technetium-99 (^{99m}Tc), while DOTA typically binds gallium or lutetium radio isotopes. One example of a linker comprising a 6-hydrazinylnicotinamide chelating moiety is

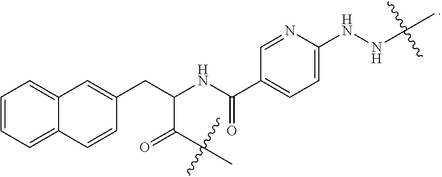 [SMILES: *NNc1ccc(C(=O)NC(Cc2ccc3ccccc3c2)C(*)=O)cn1]

### A

In the compounds described herein, A denotes a ligand for a therapeutic and/or diagnostic agent. Preferably, the therapeutic and/or diagnostic agent is a radionuclide.

Typically the ligand is a chelator for the therapeutic and/or diagnostic agent. The chelator may be bi-, tri-, tetra-, penta-, hexa-, septa- or octa-dentate.

In some embodiments, the ligand is selected from the group consisting of: TMT (6,6″-bis[N,N″,N′″-tetra(carboxymethyl)aminomethyl)-4′-(3-amino-4-methoxyphenyl)-2,2′:6′,2″-terpyridine), DOTA (1,4,7,10-tetraazacyclododecane-N,N′,N″,N″′-tetraacetic acid, also known as tetraxetan), TCMC (the tetra-primary amide of DOTA), DO3A (1,4,7,10-Tetraazacyclododecane-1,4,7-tris(acetic acid)-10-(2-thioethyl)acetamide), CB-DO2A (4,10-bis(carboxymethyl)-1,4,7,10-tetraazabicyclo[5.5.2]tetradecan), NOTA (1,4,7-triazacyclononane-triacetic acid) Diamsar (3,6,10,13,16,19-hexaazabicyclo[6.6.6]eicosane-1,8-diamine), DTPA (Pentetic acid or diethylenetriaminepentaacetic acid), CHX-A″-DTPA ([(R)-2-Amino-3-(4-isothiocyanatophenyl)propyl]-trans-(S,S)-cyclohexane-1,2-diamine-pentaacetic acid), TETA (1,4,8,11-tetraazacyclotetradecane-1,4,8,11-tetraacetic acid), Te2A (4,11-bis(carboxymethyl)-1,4,8,11-tetraazabicyclo[6.6.2]hexadecane), HBED (N,N-bis(2-Hydroxybenzyl)ethylenediamine-N,N-diacetic acid), DFO (Desferrioxamine), and analogues or derivatives thereof such as DFO* and DFOsq (DFO-squaramide), HYNIC (6-hydrazinonicotinamide),and HOPO (3,4,3-(LI-1,2-HOPO), or other ligand as described herein, or a derivative thereof. Suitable derivatives include modification to enable covalent linking to the rest of the molecule and may include functional group interconversion, such as the presence of an amide in place of a carboxyl group.

In some embodiments, the diagnostic or therapeutic agent is a radioisotope (or radionuclide). Examples of suitable isotopes include: actinium-225 (²²⁵Ac), astatine-211 (²¹¹At), bismuth-212 and bismuth-213 (²¹²Bi, ²¹³Bi), copper-64 and copper-67 (⁶⁴Cu, ⁶⁷Cu), gallium-67 and gallium-68 (⁶⁷Ga and ⁶⁸Ga), indium-111 (¹¹¹In), iodine-123, -124, -125 or -131 (¹²³I, ¹²⁴I, ¹²⁵I, ¹³¹I) (¹²³I), lead-212 (²¹²Pb), lutetium-177 (¹⁷⁷Lu), radium-223 (²²³Ra), samarium-153 (¹⁵³Sm), scandium-44 and scandium-47 (⁴⁴Sc, ⁴⁷Sc), strontium-90 (⁹⁰Sr), technetium-99 (^{99m}Tc), yttrium-86 and yttrium-90 (⁸⁶Y, ⁹⁰Y), zirconium-89 (⁸⁶Zr).

The skilled person will be aware of the therapeutic or diagnostic potential of these radionuclides. As used herein the term “diagnostic radionuclide” or “diagnostic radio isotope” refer to a radionuclide useful in diagnostic methods, typically capable of use as a contrast agent for an imaging technique. As used herein the term “therapeutic radionuclide” refers to a radionuclide useful in therapy, and typically possessing post-administration cytotoxic activity.

It will be appreciated that certain of the ligands described herein have greater affinity for one or more of the radio isotopes described herein. The skilled address will readily be able to determine what ligand should be selected for complexing a particular radionuclide, and also given a particular ligand what radionuclide should be selected for complexation. The skilled addressee will also be able to determine which radionuclides may be used for therapy and which may be used for diagnosis. For example, complexes of lutetium-177 (¹⁷⁷Lu) are typically therapeutic agents, while complexes of technetium-99 (^{99m}Tc), gallium-67 and gallium-68 (⁶⁷Ga and ⁶⁸Ga) are typically diagnostic agents.

In some embodiments, A is selected from DOTA, TETA, HBED, HYNIC and

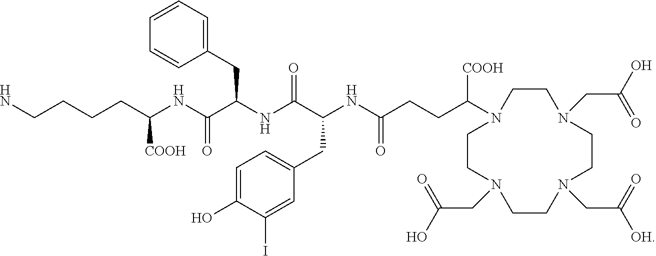

In some embodiments, the compound of formula (I) is selected from:

| Compound | |
|---|---|
| ID | Structure |
| HYNIC- iPSMA | 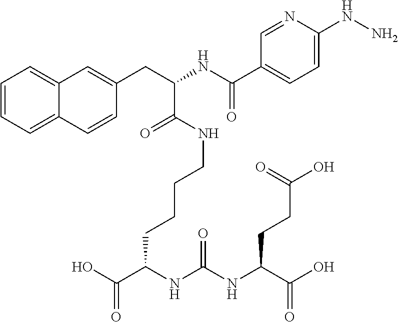 [SMILES: NNc1ccc(C(=O)N[C@@H](Cc2ccc3ccccc3c2)C(=O)NCCCC[C@H](NC(=O)N[C@@H](CCC(=O)O)C(=O)O)C(=O)O)cn1] |
| DOTA- HYNIC- iPSMA | 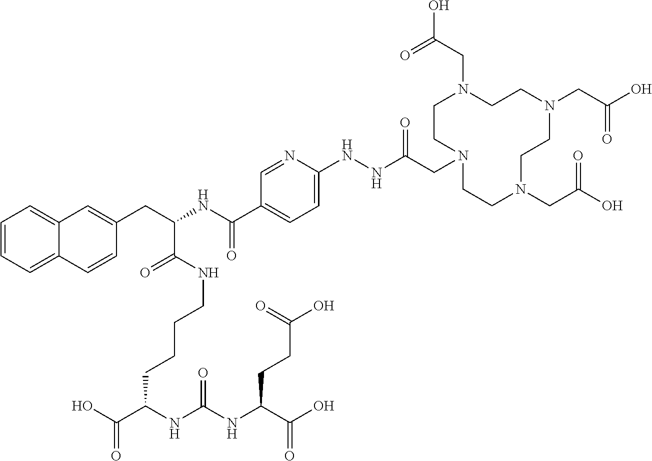 [SMILES: O=C(O)CC[C@H](NC(=O)N[C@@H](CCCCNC(=O)[C@H](Cc1ccc2ccccc2c1)NC(=O)c1ccc(NNC(=O)CN2CCN(CC(=O)O)CCN(CC(=O)O)CCN(CC(=O)O)CC2)nc1)C(=O)O)C(=O)O] |
| PSMA-11 | 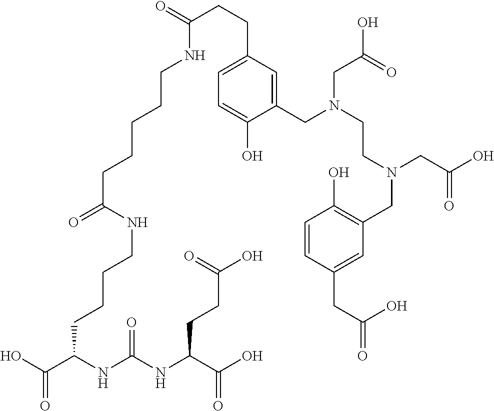 [SMILES: O=C(O)CC[C@H](NC(=O)N[C@@H](CCCCNC(=O)CCCCCNC(=O)CCc1ccc(O)c(CN(CCN(CC(=O)O)Cc2cc(CC(=O)O)ccc2O)CC(=O)O)c1)C(=O)O)C(=O)O] |
| PSMA-617 | 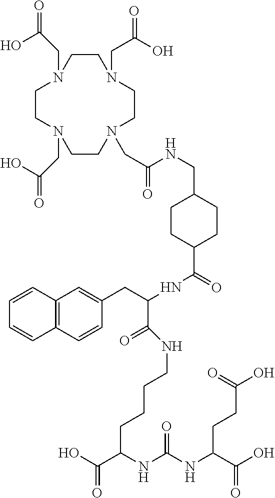 [SMILES: O=C(O)CCC(NC(=O)NC(CCCCNC(=O)C(Cc1ccc2ccccc2c1)NC(=O)C1CCC(CNC(=O)CN2CCN(CC(=O)O)CCN(CC(=O)O)CCN(CC(=O)O)CC2)CC1)C(=O)O)C(=O)O] |
| PSMA I&T | 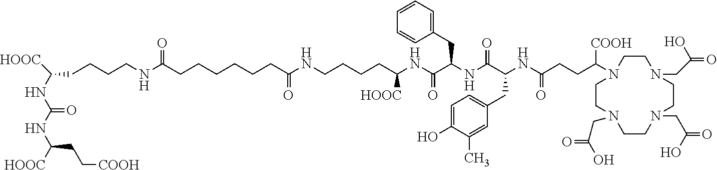 [SMILES: Cc1cc(C[C@@H](NC(=O)CCC(C(=O)O)N2CCN(CC(=O)O)CCN(CC(=O)O)CCN(CC(=O)O)CC2)C(=O)N[C@H](Cc2ccccc2)C(=O)N[C@H](CCCCNC(=O)CCCCCCC(=O)NCCCC[C@H](NC(=O)N[C@@H](CCC(=O)O)C(=O)O)C(=O)O)C(=O)O)ccc1O] |

or a salt thereof.

### Method Steps

In some embodiments, the method of preparing a compound of formula (I) of the invention includes the following steps:
a. reacting a compound of formula (II) with a compound of formula (III) to provide a compound of formula (IV);
b. selectively deprotecting the compound of formula (IV) to provide a compound of formula (V);
c. reacting the compound of formula (V) with a compound of formula (VI), formula (VII) or formula (VIII) followed by deprotection and cleavage from the solid support to provide the compound of formula (I).

As with standard solid phase synthesis, the method of the invention comprises a series of coupling (eg reacting steps a and c) followed by deprotection steps, and ends with cleavage from the solid support.

### Reacting Steps

The reacting steps of this method may be carried out using standard solid phase synthesis methodology. In some embodiments, standard Fmoc-compatible conditions are used. Suitable Fmoc-compatible conditions are described in Chen, W. C. and White, P. D. ‘Fmoc Solid Phase Peptide Sythesis: A Practical Approach’ 2000 (Oxford University Press; Hames, B. D. (Ed.)) ISBN 0199637245, which is entirely incorporated by reference.

Suitable conditions typically include contacting the amine reactant with a carboxyl reactant presented as an appropriately substituted carboxylic acid (typically in an activated form such as acid chloride, mixed anhydride, etc.) or in the presence of one or more coupling reagent(s), such as diisopropylcarbodiimide (DIC), dicyclohexyl carbodiimide (DCC), hydroxybenzotiazole (HOBt, available as Oxyma Pure), 1-[Bis(dimethylamino)methylene]-1H-1,2,3-triazolo[4,5-b]pyridinium 3-oxide hexafluorophosphate (HATU), 2-(1H-benzotriazol-1-yl)-1,1,3,3-tetramethyluronium hexafluorophosphate (HBTU) etc., optionally together with a catalyst such as 4-dimethylaminopyridine (DMAP)) to form an amide bond. The reaction may also be carried out at elevated temperature achieved either through heating (eg to a temperature of about 30-60° C.) or through microwave irradiation. In some embodiments, similar conditions may be used to react the compound of formula (II) and formula (III), and react the compound of formula (V) with a compound of formula (VI)-(VIII).

To facilitate the use of standard solid phase peptide coupling conditions, the solid support may be any suitable solid phase resin. In some embodiments, the solid phase is a 2-chloro-trityl resin (CT resin). Accordingly, in some embodiments, the compounds of the invention are linked to the solid support through a 2-chlorotrityl moiety. While formulas (II)-(V) depict a single molecule bound to the solid support, each solid support is typically loaded with a plurality of such compounds. Optionally, the solid phase synthetic methods described herein may comprise a capping step (for example with acetic anhydride or other activated capping agent) after each reacting step to cap any unprotected sites and prevent synthesis of compounds potentially lacking a single moiety, which may be hard to separate from the desired product.

### Deprotection

Conditions to carry out the deprotection step(s) depend on the protecting group selected. Typically, deprotection includes treating the protected compound with an acid or base. There are usually multiple conditions possible to cleave a protecting group and the skilled person will be able to determine appropriate conditions depending on the solid support selected, the protecting group to be removed and the remaining functionality in the compound.

Selective deprotecting steps require the selection of conditions able to cleave a protecting group in the presence of one or more further protecting groups, e.g. the deprotection of PG¹ in the presence of PG² and PG³. Accordingly, in some embodiments, PG¹ is not the same as PG² or PG³. While PG² and PG³ may be the same or different, preferably they are the same protecting group which is different to PG¹. In some embodiments, the selective deprotecting step is carried out at temperatures below ambient, such as at 0° C. or less.

In some embodiments, PG¹ is an alloc (allyloxycarbonyl) protecting group.

In some embodiments, PG² is a tert-butyl protecting group.

In some embodiments, PG³ is a tert-butyl protecting group.

In some embodiments, PG¹ is alloc and PG² and PG³ are each tert-butyl.

### Cleavage

Conditions required to cleave the growing compounds from the solid support depend on the solid support selected. Typically, cleavage conditions include exposing the resin to acid (eg trifluoroacetic acid (TFA)) or base optionally at an elevated temperature. In some embodiments, the conditions to cleave the compound from the solid support are also capable of global deprotection of any remaining protecting groups within the compound.

In any of the reaction steps involving compounds covalently bound to the solid support described herein, reagents may be used in a molar excess relative to the bound species, typically molar excesses of at least about 1.1 molar equivalents, 5 molar equivalents, 10 molar equivalents, 25 molar equivalents, 50 molar equivalents, 100 molar equivalents, 1000 molar equivalents or more. The molar excess may be between any of these values, for example, from about 1.1 to about 1000 molar equivalents, or about 5 to about 100 molar equivalents.

### Intermediates

The invention also relates to key synthetic intermediates of this method and the methods of preparing the intermediates, such as compounds of formulas (II)-(X).

Each variable in the compounds of formulas (II)-(X) may be as defined for the corresponding variable of formula (I).

In some embodiments, there is provided a compound of formula (II) or a salt thereof

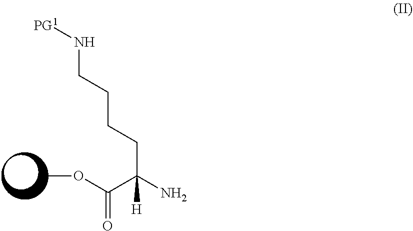

wherein:
[symbol] denotes a solid support; and
PG¹ is an amine protecting group.

In some embodiments, the solid support is a solid support resin.

In some embodiments, the solid support is a 2-chlorotrityl resin.

In some embodiments, PG¹ is alloc.

In some embodiments, there is provided a compound of formula (III) or a salt thereof

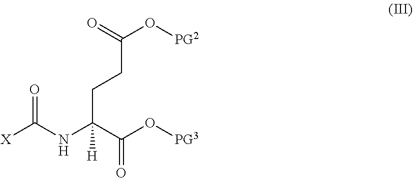

wherein:
X is —OH or a carbonyl activating group; and
PG² and PG³ are independently carboxyl protecting groups.

In some embodiments, X is a carbonyl activating group selected from a halide (such as Cl, Br), an activated ester (such as a pentafluorophenyl ester, N-hydroxysuccinamide ester, etc.), a mixed anhydride (such as acetyl and the like) and so on. Techniques to prepare suitable activated carboxyl groups are known in the art. Coupling the activated carboxyl of the compound of formula (III) may be mediated by standard coupling conditions as described herein (eg. in the presence of a coupling agent and optionally a catalyst), however, as the carboxyl group is activated no coupling agent may be necessary.

In some embodiments, X is —OH. In these embodiments, the reaction of the compound of formula (II) and formula (III) is mediated by a coupling reagent, which typically provides an activated form of the carboxyl group in situ.

In some embodiments, PG² and PG³ are the same protecting group. Typically, PG² and PG³ are selected to be different to PG¹ in the compound of formula (II) to provide an orthogonal protecting group strategy and enabling their selective deprotection. In some embodiments, PG² and PG³ are each a tert-butyl group.

In one aspect, the invention provides a compound of formula (IV) or a salt thereof:

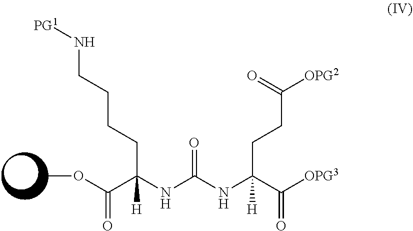

wherein
[symbol] denotes a solid support;
PG¹ is an amine protecting group; and
PG² and PG³ are independently carboxyl protecting groups.

The solid support, PG¹, PG² and PG³ may be as defined herein.

In one aspect, the invention provides a compound of formula (V) or a salt thereof:

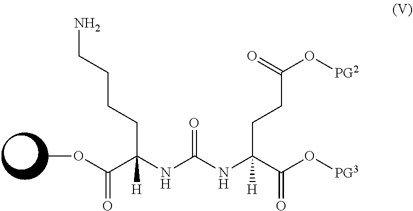

wherein
[symbol] denotes a solid support;
PG² and PG³ are independently carboxyl protecting groups.

In some embodiments, the solid support may be any suitable solid support described herein, including any solid support defined in formula (I) or (II).

In some embodiments, PG² and PG³ are as defined for formula (III). In some embodiments, PG² and PG³ are each tert-butyl.

In one aspect, the invention provides a compound of formula (V′) or a salt thereof:

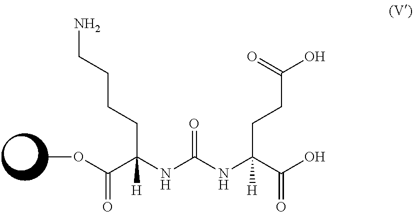 [SMILES: COC(=O)[C@H](CCCCN)NC(=O)N[C@@H](CCC(=O)O)C(=O)O]

wherein
[symbol] denotes a solid support;

In some embodiments, the solid support may be any suitable solid support described herein, including any solid support defined in formula (I) or (II).

In some embodiments, there is provided a compound of formula (VI), (VII) and (VIII) or a salt thereof:

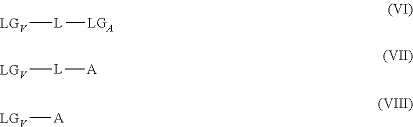

wherein
L is a bifunctional linker or a protected form of a bifunctional linker;
A is a ligand for a therapeutic or diagnostic agent or a protected form thereof;
LG_{V} is a group cleavable in the reaction with the compound of formula (V)
LG_{A} is H or a group cleavable in a subsequent step reacting moiety L with a synthon of moiety A.

In some embodiments, LG_{V} and LG_{A} are independently selected from an amine protecting group, a carboxyl protecting group and a carboxamide protecting group.

In some embodiments, LG_{A} is Fmoc.

In some embodiments, LG_{V} is —OH or a carboxyl-activating group. LG_{V} may be any carboxyl-activating group described herein.

In some embodiments, L is any bifunctional linker described herein or a protected form thereof. The person skilled in the art will be able to select appropriate protecting groups depending on the reactive functional groups present in the desired ligand. For example, carboxylic acid moieties in a ligand may be protected as tert-butyl esters.

The compounds of formulas (VI)-(VIII) are synthons for introducing the —L-A moiety of the compound of formula (I) by reaction with the deprotected lysine side chain amine of the compound of formula (V), either directly (formulas (VII) and (VIII)) or indirectly (formula (VI)). Accordingly, in some embodiments, the methods may comprise reacting the compound of formula (V) with any suitable compound to install the —L-A moiety of the compound of formula (I), such as compounds of formulas (VI)-(VIII).

The compounds of formula (VII) and (VIII) each represent a synthon for the direct introduction of the —L-A moiety as defined for formula (I).

The compound of formula (VI) represent a synthon introducing the —L-A moiety in a step-wise manner. Accordingly, after reacting the compound of formula (V) with a compound of formula (VI), the methods may further comprise, prior to deprotection and cleavage from the solid support, reacting a compound of formula (IX) with LG_{L}-A, wherein LG_{L} is H or a group cleavable to form a bond from moiety L to moiety A, and A is a ligand for a therapeutic or diagnostic agent or a protected form thereof

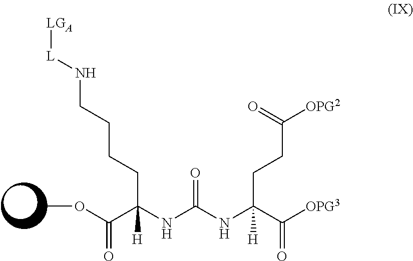

wherein L and LG_{A} are as defined in formula (VI) and PG² and PG³ are as defined for formula (V).

In some embodiments, LG_{L}-A is selected from a compound of formula (XI) and a compound of formula (XII)

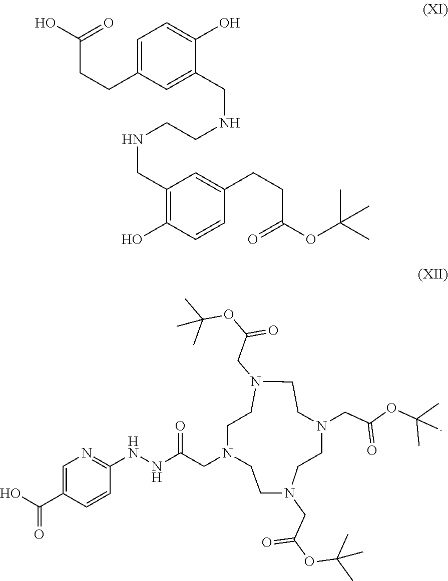

The compounds of formulas (XI) and (XII) may be prepared by any suitable methods in the art, including those described herein.

In another aspect, also provided is a method of preparing a compound of formula (I) as defined in claim 1, the method comprising subjecting a compound of formula (X)

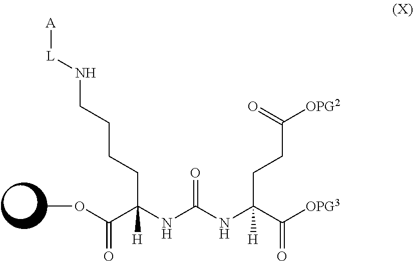

wherein
[symbol] denotes a solid support; and
L is a covalent bond or a bifunctional linker or a protected form thereof;
A is a ligand for a diagnostic or therapeutic agent or a protected form thereof; and
PG² and PG³ are independently H or a carboxyl protecting group,
in one or more steps to conditions suitable to effect cleavage from the solid support and optionally deprotecting the compound.

The methods of the invention allow for the facile solid phase synthesis of a range of conjugates to the GUL-derived PSMA targeting moiety resin bound in the compound of formula (V).

Also provided is a method of preparing a compound of formula (IV) or a salt thereof, the method comprising reacting (or coupling) a compound of formula (II) and a compound of formula (III). Advantageously, the compound of formula (IV) may be preferable for storage on resin, and may be selectively deprotected to provide a compound of formula (V) when required.

Also provided is a method of preparing a compound of formula (V) or a salt thereof, the method comprising selective deprotection of PG¹ in the compound of formula (IV) to provide the compound of formula (V).

Also provided is a method of preparing a compound of formula (V) or a salt thereof, the method comprising reacting (or coupling) a compound of formula (II) and a compound of formula (III) and selectively cleaving moiety PG¹.

Also provided is a method of preparing a compound of formula (V′) or a salt thereof, the method comprising reacting (or coupling) a compound of formula (II) and a compound of formula (III) and selectively cleaving moieties PG¹, PG² and PG³. In these methods, cleavage of PG¹, PG² and PG³ is carried out under conditions that are not suitable for cleaving the covalent bond connecting the compound to the solid support. Cleavage of moieties PG¹, PG² and PG³ may be carried out in a single deprotection step under any suitable conditions described herein. In some embodiments, PG¹ is deprotected in a separate step to PG² and PG³. The skilled person will be able to select a suitable amino protecting group at PG¹ and at PG² and PG³ and will understand suitable conditions for their removal.

Also provided is a method of preparing a compound of formula (I) or a salt thereof:

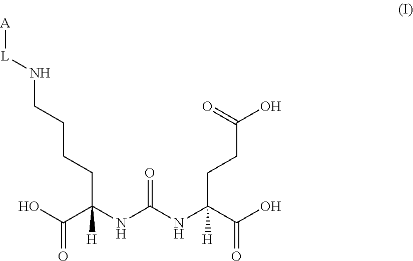

wherein
L is a covalent bond or a bifunctional linker; and
A is a ligand for a diagnostic or therapeutic agent;
the method comprising:
   reacting the compound of formula (V′) as defined herein with a compound of formula (VI), formula (VII) or formula (VIII) followed by and cleavage from the solid support to provide the compound of formula (I)

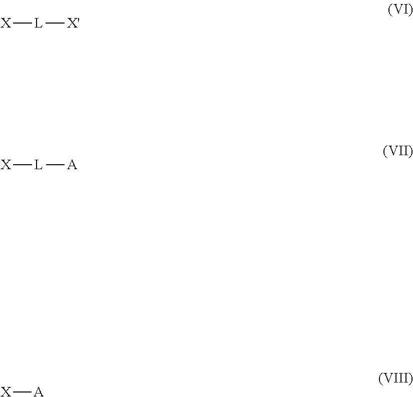

wherein
L is as defined for formula (I), with the proviso that in formula (VI), L is a bifunctional linker;
A is as defined for formula (I)
X is a group cleavable in the reaction with the compound of formula (V′)
X′ is H or a group cleavable in a subsequent step reacting moiety L with a synthon of moiety A. It will be appreciated that in some of embodiments of these methods of preparing compounds of formula (I) involving reaction of compounds of formula (V′) differ to those described herein for preparing compounds of formula (I) involving reaction of compounds of formula (V) in terms of the timing of deprotection steps (eg in reactions involving a compound of formula (V′) cleavage of PG² and PG³ may be carried out prior to reaction with a compound of formula (VI), (VII) and (VIII)), so where appropriate conditions/reagents for steps in these methods may therefore be as described herein for any methods of preparing compounds of formula (I).

### Methods of Preparing the Compound of Formula (XII)

Also provided herein are methods of preparing a compound of formula (XII) or a salt thereof

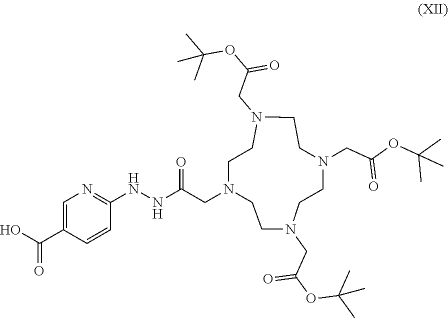 [SMILES: CC(C)(C)OC(=O)CN1CCN(CC(=O)NNc2ccc(C(=O)O)cn2)CCN(CC(=O)OC(C)(C)C)CCN(CC(=O)OC(C)(C)C)CC1]

the method comprising selectively deprotecting PG⁴ of a compound of formula (XIII)

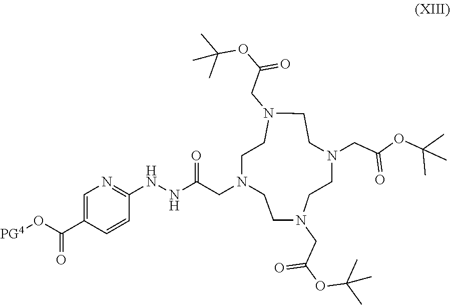

wherein
PG⁴ is a carboxyl protecting group orthogonal with a ter-butyl ester protecting group.

In some embodiments, PG⁴ is a benzyl protecting group. In this context, the term “benzyl protecting group” will be understood to mean that the protecting group is a benzyl group in which the benzyl ring is optionally substituted (ie the benzyl ring may be unsubstituted or substituted) with one or more substituents. Optional substituents of the phenyl moiety of the benzyl group include hydroxy, halo (eg chloro, bromo, fluoro or iodo), and optionaly substituents of the methylene moiety of the benzyl include further optionally substituted phenyl substituents which may each be the same or different. Preferably optional substituents of the pheyl moiety may be at the para-position relative to the methylene moiety of the benzyl group. In preferred embodiments, PG⁴ is unsubstituted benzyl.

Conditions for deprotecting PG⁴ may be any suitable conditions known in the art for cleaving the selected protecting group, including those described herein, provided the conditions are orthogonal with a tert-butyl ester protecting group. Typically, deprotection includes treating the compound of formula (XIII) with a base or subjecting the compound of formula (XIII) to hydrogenolysis. There are usually multiple conditions possible to cleave a protecting group and the skilled person will be able to determine appropriate conditions depending on the protecting group to be removed and the remaining functionality in the compound.

In some embodiments, including embodiments wherein PG⁴ is a benzyl protecting group, the deprotecting step comprises hydrogenolysis of PG⁴. Conditions for hydrogenolysis may be any suitable conditions known in the art, including those described herein. Examples of suitable conditions include hydrogenation in the presence of a suitable catalyst such as a nickel, palladium or platinum catalyst. The hydrogenation may be performed under atmospheric conditions (1 atm H₂) or elevated pressure (eg by using a pressurised vessel such as a Parr hydrogenator, or a flow reactor).

The compound of formula (XIII) may be prepared by reacting a compound of formula (XIV)

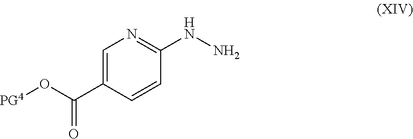

wherein
PG⁴ is a carboxyl protecting group orthogonal with a tert-butyl ester protecting group, preferably PG⁴ is a benzyl protecting group, more preferably PG⁴ is benzyl;
with a compound of formula (XV)

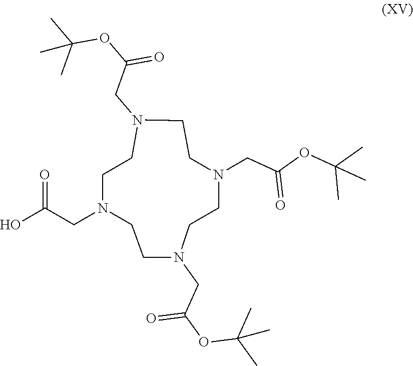 [SMILES: CC(C)(C)OC(=O)CN1CCN(CC(=O)O)CCN(CC(=O)OC(C)(C)C)CCN(CC(=O)OC(C)(C)C)CC1]

Conditions for the reacting step may be any suitable conditions known in the art, including those described herein.

The compound of formula (XIV) may be prepared by cleaving the Boc protecting group of a compound of formula (XVI)

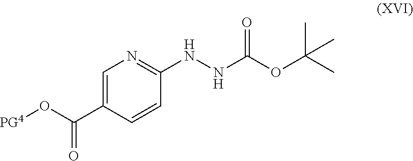

wherein
PG⁴ is a carboxyl protecting group orthogonal with a tert-butyl ester protecting group, preferably PG⁴ is a benzyl protecting group, more preferably PG⁴ is benzyl.

Conditions for cleaving the Boc group may be any suitable conditions known in the art, including those described herein. In some embodiments, the cleaving step comprises contacting the compound of formula (XVI) to an acid, such as hydrochloric acid.

The compound of formula (XVI) may be prepared by reacting the carboxylic acid group of a compound of formula (XVII)

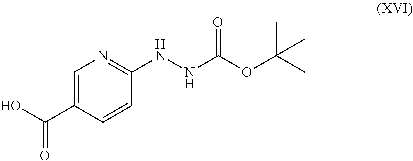 [SMILES: CC(C)(C)OC(=O)NNc1ccc(C(=O)O)cn1]

with a synthon of PG⁴.

Reagents and conditions for introducing PG⁴ may be any suitable conditions known in the art for installing the selected protecting group, including those described herein. There are usually multiple conditions possible to introduce a protecting group and the skilled person will be able to determine appropriate conditions depending on the protecting group to be installed and the remaining functionality in the compound.

In embodiments where PG⁴ is benzyl, the compound of formula (XVI) may be prepared by reacting the a compound of formula (XVII) with a compound of formula (XVIII)

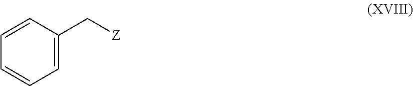

wherein Z is a suitable leaving group such as OH or a halide, preferably chloride or bromide.

Accordingly, in some embodiments, the method of preparing the compound of formula (XII) comprises one or more of the following steps:
reacting a compound of formula (XVII) with a synthon of PG⁴ (eg a compound of formula (XVIII)) to provide a compound of formula (XVI)

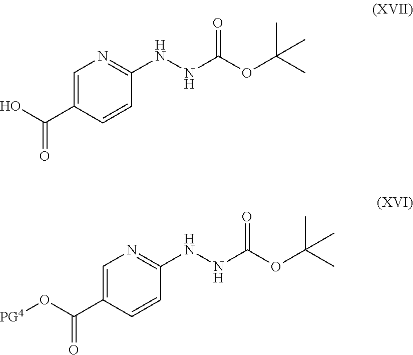

cleaving the Boc protecting group of the compound of formula (XVI) to provide a compound of formula (XIV)

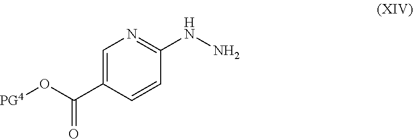

reacting the compound of formula (XIV) with a compound of formula (XV) to provide a compound of formula (XIII)

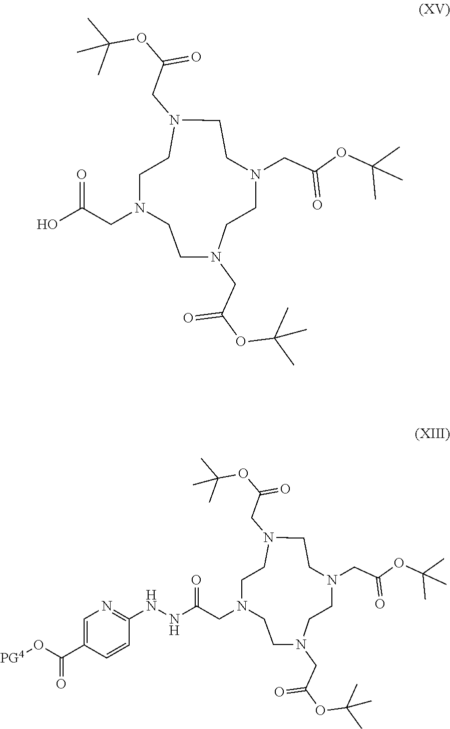

selectively deprotecting PG⁴ (eg by hydrogenolysis) of the compound of formula (XIII) to provide a compound of formula (XII)

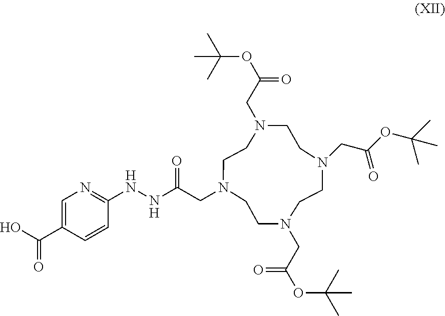 [SMILES: CC(C)(C)OC(=O)CN1CCN(CC(=O)NNc2ccc(C(=O)O)cn2)CCN(CC(=O)OC(C)(C)C)CCN(CC(=O)OC(C)(C)C)CC1]

wherein PG⁴ is a carboxyl protecting group orthogonal with a tent-butyl ester protecting group, preferably PG⁴ is a benzyl protecting group, more preferably PG⁴ is benzyl.

The invention also relates to key synthetic intermediates of this method and the methods of preparing the intermediates, such as compounds of formulas (XIII), (XIV) and (XVI).

The compound of formula (XII) prepared by this method may be used in the method of preparing the compound of formula (I) described herein. Accordingly, the compound of formula (XII), and synthetic intermediates used in the preparation of the compound of formula (XII), may alternatively be referred to as intermediate compounds.

It will be understood that the invention disclosed and defined in this specification extends to all alternative combinations of two or more of the individual features mentioned or evident from the text or drawings. All of these different combinations constitute various alternative aspects of the invention.

### Alternative Forms of Compounds

The salts of the compounds described herein (including compounds of any of formulas (I), (II), (Ill), (IV), (V), (V′), (VI), (VII), (VIII), (IX), (X), collectively referred to herein as compound of formulas (I)-(X)) are preferably pharmaceutically acceptable, but it will be appreciated that non-pharmaceutically acceptable salts also fall within the scope of the present disclosure, since these may be useful as intermediates in the preparation of pharmaceutically acceptable salts.

The term “pharmaceutically acceptable” may be used to describe any salt, solvate, tautomer, N-oxide, stereoisomer and/or prodrug thereof, or any other compound which upon administration to a subject, is capable of providing (directly or indirectly) a compound of Formula (I) or an active metabolite or residue thereof.

Suitable pharmaceutically acceptable salts include, but are not limited to, salts of pharmaceutically acceptable inorganic acids such as hydrochloric, sulphuric, phosphoric, nitric, carbonic, boric, sulfamic, and hydrobromic acids, or salts of pharmaceutically acceptable organic acids such as acetic, propionic, butyric, tartaric, maleic, hydroxymaleic, fumaric, malic, citric, lactic, mucic, gluconic, benzoic, succinic, oxalic, phenylacetic, methanesulphonic, toluenesulphonic, benzenesulphonic, salicylic, sulphanilic, aspartic, glutamic, edetic, stearic, palmitic, oleic, lauric, pantothenic, tannic, ascorbic and valeric acids.

Base salts include, but are not limited to, those formed with pharmaceutically acceptable cations, such as sodium, potassium, lithium, calcium, magnesium, zinc, ammonium, alkylammonium such as salts formed from triethylamine, alkoxyammonium such as those formed with ethanolamine and salts formed from ethylenediamine, choline or amino acids such as arginine, lysine or histidine. General information on types of pharmaceutically acceptable salts and their formation is known to those skilled in the art and is as described in general texts such as “Handbook of Pharmaceutical salts” P. H. Stahl, C. G. Wermuth, 1st edition, 2002, Wiley-VCH.

In the case of compounds that are solids, it will be understood by those skilled in the art that the inventive compounds, agents and salts may exist in different crystalline or polymorphic forms, all of which are intended to be within the scope of the present invention and specified formulae.

Formulas described herein are intended to cover, where applicable, solvated as well as unsolvated forms of the compounds. Thus, for example, Formula (I) includes compounds having the indicated structures, including the hydrated or solvated forms, as well as the non-hydrated and non-solvated forms.

The compounds of Formulas (I) or salts, tautomers, N-oxides, polymorphs or prodrugs thereof may be provided in the form of solvates. Solvates contain either stoichiometric or non-stoichiometric amounts of a solvent, and may be formed during the process of crystallization with pharmaceutically acceptable solvents such as water, alcohols such as methanol, ethanol or isopropyl alcohol, DMSO, acetonitrile, dimethyl formamide (DMF), acetic acid, and the like with the solvate forming part of the crystal lattice by either non-covalent binding or by occupying a hole in the crystal lattice. Hydrates are formed when the solvent is water, alcoholates are formed when the solvent is alcohol. Solvates of the compounds of the present invention can be conveniently prepared or formed during the processes described herein. In general, the solvated forms are considered equivalent to the unsolvated forms for the purposes of the compounds and methods provided herein.

Basic nitrogen-containing groups may be quarternised with such agents as C₁₋₆ alkyl halide, such as methyl, ethyl, propyl, and butyl chlorides, bromides and iodides; dialkyl sulfates like dimethyl and diethyl sulfate; and others.

Nitrogen containing groups may also be oxidised to form an N-oxide.

The compound of Formula (I) or salts, tautomers, N-oxides, solvates and/or prodrugs thereof that form crystalline solids may demonstrate polymorphism. All polymorphic forms of the compounds, salts, tautomers, N-oxides, solvates and/or prodrugs are within the scope of this invention and may be used in the methods of the invention.

The compound of Formulas (I)-(X) may demonstrate tautomerism. Tautomers are two interchangeable forms of a molecule that typically exist within an equilibrium. Any tautomers of the compounds are to be understood as being within the scope of the invention and may be used in the methods of the invention.

The compounds of Formulas (I)-(X) may contain one or more stereocentres. All steoisomers of these compounds are within the scope of the invention. Stereoisomers include enantiomers, diastereomers, geometric isomers (E and Z olephinic forms and cis and trans substitution patterns) and atropisomers. In some embodiments, the compound is a stereoisomerically enriched form of the compound of formula (I) at any stereocentre. The compound may be enriched in one stereoisomer over another by about 60, 70, 80, 90, 95, 98 or 99%.

The compound of Formula (I)-(X) or its salts, tautomers, solvates, N-oxides, polymorphs and/or stereoisomers, may be isotopically enriched with one or more of the isotopes of the atoms present in the compound. For example, the compound may be enriched with one or more of the following minor isotopes: ²H, ³H, ¹³C, ¹⁴C, ¹⁵N and/or ¹⁷O. An isotope may be considered enriched when its abundance is greater than its natural abundance.

A “prodrug” is a compound that may not fully satisfy the structural requirements of the compounds provided herein, but is modified in vivo, following administration to a subject or patient, to produce a compound of formula (I) provided herein. For example, a prodrug may be an acylated derivative of a compound as provided herein. Prodrugs include compounds wherein hydroxy, carboxy, amine or sulfhydryl groups are bonded to any group that, when administered to a mammalian subject, cleaves to form a free hydroxy, carboxy, amino, or sulfhydryl group, respectively. Examples of prodrugs include, but are not limited to, acetate, formate, phosphate and benzoate derivatives of alcohol and amine functional groups within the compounds provided herein. Prodrugs of the compounds provided herein may be prepared by modifying functional groups present in the compounds in such a way that the modifications are cleaved in vivo to generate the parent compounds.

Prodrugs include compounds wherein an amino acid residue, or a polypeptide chain of two or more (eg, two, three or four) amino acid residues which are covalently joined to free amino, and amido groups of compounds of Formula (I). The amino acid residues include the 20 naturally occurring amino acids commonly designated by three letter symbols and also include, 4-hydroxyproline, hydroxylysine, demosine, isodemosine, 3-methylhistidine, norvlin, beta-alanine, gamma-aminobutyric acid, citrulline, homocysteine, homoserine, ornithine and methionine sulfone. Prodrugs also include compounds wherein carbonates, carbamates, amides and alkyl esters which are covalently bonded to the above substituents of Formula (I) through the carbonyl carbon prodrug sidechain.

### Therapeutic, Diagnostic and Theranostic Agents

The compounds of formula (I) prepared by the methods of the invention are precursors for a therapeutic and/or diagnostic agent. Conversion of the compounds of formula (I) into the therapeutic and/or diagnostic agent, typically comprises exposing the compound of formula (I) to a therapeutic and/or diagnostic agent (such as a radionuclide) to form a complex.

In some embodiments, the compound of formula (I) prepared by a method of the invention forms a complex with a therapeutic radio isotope and thus forms a therapeutic agent.

In some embodiments, the compound of formula (I) prepared by a method of the invention forms a complex with a radio isotope for use in imaging, and thus forms a diagnostic agent.

In some embodiments, the compound of formula (I) prepared by a method of the invention forms a complex with one or more radio isotopes for use in therapy and imaging, and thus forms a theranostic agent.

The compound of formula (I) may be formulated as a pharmaceutical composition for any appropriate route of administration including, for example, or al, rectal, nasal, vaginal, topical (including transdermal, buccal, ocular and sublingual), parenteral (including subcutaneous, intraperitoneal, intradermal, intravascular (for example, intravenous), intramuscular, spinal, intracranial, intrathecal, intraocular, periocular, intraorbital, intrasynovial and intraperitoneal injection, intracisternal injection as well as any other similar injection or infusion techniques), inhalation, insufflation, infusion or implantation techniques (e.g., as sterile injectable aqueous or non-aqueous solutions or suspensions).

The pharmaceutical compositions may be formulated together with one or more pharmaceutically acceptable components (e.g. excipients, diluents and/or carriers). Examples of components are described in Martindale—The Extra Pharmacopoeia (Pharmaceutical Press, London 1993), and Remington: The Science and Practice of Pharmacy, 21st Ed., 2005, Lippincott Williams & Wilkins. All methods include the step of bringing the active ingredient, for example a compound defined by Formula (I), or a pharmaceutically acceptable salt or prodrug thereof, into association with the carrier which constitutes one or more accessory ingredients. When the therapeutic agent or diagnostic agent is a radionuclide, the method may further comprise combining the pharmaceutical composition with the radionuclide to form a complex in a formulation suitable for administration to a subject. In general, the pharmaceutical compositions are prepared by uniformly and intimately bringing the object compound, for example a compound defined by Formula (I), or a pharmaceutically acceptable salt or prodrug thereof, into association with a liquid carrier or a finely divided solid carrier or both, and then, if necessary, shaping the product into the desired formulation. In the pharmaceutical composition the object compound is included in an amount sufficient to produce the desired effect.

### Methods of Treatment

Also described are methods of treating prostate cancer, comprising administering to a subject in need thereof an effective amount of a complex of a therapeutic radio isotope and a compound of formula (I) prepared by a method of the invention or a pharmaceutically acceptable salt thereof.

Also described is use of a compound of formula (I) of the invention or a pharmaceutically acceptable salt thereof in the preparation of a medicament for treating prostate cancer.

The prostate cancer may be any form of prostate cancer expressing PSMA. The prostate cancer may express as a prostate cancer tumour. In some embodiments, the prostate cancer is castration-resistant prostate cancer.

In the context of this specification the term “administering” and variations of that term including “administer” and “administration”, includes contacting, applying, delivering or providing a compound or composition of the invention to an organism, or a surface by any appropriate means.

For the treatment of prostate cancer, the dose of the biologically active complex according to the invention may vary within wide limits and may be adjusted to individual requirements. Active compounds according to the present invention are generally administered in a therapeutically effective amount. The daily dose may be administered as a single dose or in a plurality of doses. The amount of active ingredient that may be combined with the carrier materials to produce a single dosage form will vary depending upon the subject treated and the particular mode of administration.

It will be understood, however, that the specific dose level for any particular subject will depend upon a variety of factors including the activity of the specific compound employed, the age, body weight, general health, sex and diet of the subject, time of administration, route of administration, and rate of excretion, drug combination (i.e. other drugs being used to treat the subject), and the severity of the particular disorder undergoing therapy. Such treatments may be administered as often as necessary and for the period of time judged necessary by the treating physician. A person skilled in the art will appreciate that the dosage regime or therapeutically effective amount of the compound of formula (I) to be administered may need to be optimized for each individual.

It will also be appreciated that different dosages may be required for treating different disorders. In some embodiments, an effective amount of an agent is that amount which causes a statistically significant decrease in tumour size.

The terms “treating”, “treatment” and “therapy” are used herein to refer to curative therapy, prophylactic therapy and preventative therapy. Thus, in the context of the present disclosure the term “treating” encompasses curing, ameliorating or tempering the severity of prostate cancer and/or associated diseases or their symptoms.

“Preventing” or “prevention” means preventing the occurrence of the prostate cancer or tempering the severity of the prostate cancer if it develops subsequent to the administration of the compounds or pharmaceutical compositions of the present invention. In some embodiments, preventing prostate cancer may comprise monitoring of the progress of the prostate cancer over a period of time, comprising administration of a complex of a diagnostic or theranostic radio isotope and a compound of formula (I) of the invention.

“Subject” includes any human or non-human animal. Thus, in addition to being useful for human treatment, the compounds of the present invention may also be useful for veterinary treatment of mammals, including companion animals and farm animals, such as, but not limited to dogs, cats, horses, cows, sheep, and pigs.

The compounds of the present invention may be administered along with a pharmaceutical carrier, diluent and/or excipient as described above.

In some embodiments, the compound of the invention may be administered in combination with a further active pharmaceutical ingredient (API). The API may be any that is suitable for treating prostate cancer or a symptom thereof. The compound of the invention may be co-formulated with the further API in any of the pharmaceutical compositions described herein, or the compound of the invention may be administered in a concurrent, sequential or separate manner. Concurrent administration includes administering the compound of the invention at the same time as the other API, whether coformulated or in separate dosage forms administered through the same or different route. Sequential administration includes administering, by the same or different route, the compound of the invention and the other API according to a resolved dosage regimen, such as within about 0.5, 1, 2, 3, 4, 5, or 6 hours of the other. When sequentially administered, the compound of the invention may be administered before or after administration of the other API. Separate administration includes administering the compound of the invention and the other API according to regimens that are independent of each other and by any route suitable for either active, which may be the same or different.

The methods may comprise administering the compound of Formula (I) in any pharmaceutically acceptable form. In some embodiments, the compound of Formula (I) is provided in the form of a pharmaceutically acceptable salt, solvate, N-oxide, polymorph, tautomer or prodrug thereof, or a combination of these forms in any ratio.

The methods may also comprise administering a pharmaceutical composition comprising the compound of formula (I) or a pharmaceutically acceptable salt, solvate, N-oxide, polymorph, tautomer or prodrug thereof to the subject in need thereof. The pharmaceutical composition may comprise any pharmaceutically acceptable carrier, diluent and/or excipient described herein.

The compounds of Formula (I), or a pharmaceutically acceptable salt or prodrug thereof, as defined herein, may be administered by any suitable means, for example, or ally, rectally, nasally, vaginally, topically (including buccal and sub-lingual), parenterally, such as by subcutaneous, intraperitoneal, intravenous, intramuscular, or intracisternal injection, inhalation, insufflation, infusion or implantation techniques (e.g., as sterile injectable aqueous or non-aqueous solutions or suspensions).

The compounds of the invention may be provided as pharmaceutical compositions including those for oral, rectal, nasal, topical (including buccal and sub-lingual), parenteral administration (including intramuscular, intraperitoneal, sub-cutaneous and intravenous), or in a form suitable for administration by inhalation or insufflation. The compounds of Formula (I), or a pharmaceutically acceptable salt or prodrug thereof, together with a conventional adjuvant, carrier or diluent, may thus be placed into the form of pharmaceutical compositions and unit dosages thereof, and in such form may be employed as solids, such as tablets or filled capsules, or liquids as solutions, suspensions, emulsions, elixirs or capsules filled with the same, all for or al use, or in the form of sterile injectable solutions for parenteral (including subcutaneous) use.

### Methods of Imaging, Diagnosis, Prognosis and Monitoring

Also described is a method of imaging a prostate cancer tumour, comprising administering to a subject in need thereof an effective amount of a complex of a diagnostic radio isotope and a compound of formula (I) of the invention or a pharmaceutically acceptable salt thereof, or a composition comprising the complex, and imaging the prostate cancer tumour.

Also described is a method of diagnosing, monitoring or prognosing a prostate cancer, comprising:
(a) administering to a subject in need thereof an effective amount of a complex of a diagnostic radio isotope and a compound of formula (I) of the invention or a pharmaceutically acceptable salt thereof, or a composition comprising the complex,
(b) allowing the complex to concentrate at sites in the subject where PSMA is found; and
(c) detecting the radio isotope of the complex
whereby detection of the radio isotope enables an image of the cancer (if present) to be created to thereby diagnose, monitor or prognose the prostate cancer.

In these methods of diagnosing, monitoring or prognosing a prostate cancer, the detecting step may comprise subjecting the subject to an imaging technique. The imaging technique may allow imaging of the prostate to determine the presence or change in concentation of radio isotope.

The skilled person will be familiar with methods for selecting suitable diagnostic agents for use with the complexes/compounds of the invention, including radioisotopes for use in radioimaging for diagnosing conditions described herein. Further, the skilled person will be familiar with imaging techniques for use in conjunction with the diagnostic reagents described herein.

In some embodiments, the diagnostic methods (including imaging methods) comprise subjecting the subject to positron emission tomography (PET) imaging, preferably immuno-PET imaging. PET imaging is a functional imaging technique applied in nuclear medicine, whereby a three-dimensional image (e.g. of functional processes) in the body is produced. The system detects pairs of gamma rays emitted indirectly by a positron-emitting radionuclide, which is introduced into the body in form of a pharmaceutical compound.

In some embodiments, the diagnostic method comprises subjecting the subject to magnetic resonance imaging (MRI), preferably wherein the nuclide of diagnostic potential is Gd.

In some embodiments, the diagnostic method of the invention may be used in combination with another diagnostic method, such as magnetic resonance imaging (MRI), radiography, ultrasound, elastography, photoacoustic imaging, tomography (including computed tomography) and echocardiography; preferably magnetic resonance imaging (MRI) and tomography (including computed tomography).

As used herein, a theranostic method is a method for the in vitro and/or in vivo visualization, identification and/or detection of tumour cells and/or metastases as well as a method of treatment of prostate cancer.

In some embodiments, the present invention includes a theranostic method that comprises:
(1) administering a diagnostically-effective amount a complex of diagnostic radio isotope and a compound of formula (I) of the present invention to a patient or subject, and
(2) administering a therapeutically-effective amount of a complex of a therapeutic radio isotope and a compound of formula (I) of the present invention to the patient or subject in need thereof.

Preferably, step 1 and step 2 are conducted sequentially and the compound in step 1 and step 2 is the same.

The methods, compounds and intermediates described herein are described by the following illustrative and non-limiting examples.

### EXAMPLES

General

Some examples described herein are based on solid phase-peptide synthesis. For these syntheses, the following apply. Some steps in the synthetic schemes may involve both a coupling step (denoted “c”) and a deprotecting step (denoted “d”). All molar equivalents (eq) were calculated based on the loading of the resin (expressed in mmol/g resin). All volumes (V) were calculated based on the weight of the functionalised resin. The volume is represented as a multiple per gram of functionalised resin. By way of example, 5 volume equivalents per gram of resin is represented as 5V. All reactions were performed at room temperature (typically about 20±5° C.). To analyse products grafted on the resin (including intermediate products), a sample of the product on the resin was cleaved using HFIP, without degradation of protecting groups (if present), and analysed without purification. Analyses performed on the cleaved product reflect the product on the resin. The analyses were performed using the following procedure: (1) mixing the sample of functionalised resin with 10 mL DCM/HFIP solution (8:2); (2) stirring the mixture using orbital stirring (200 rpm) at room temperature for 2 hours; (3) filtering the mixture (porosity 3); (4) concentrating the mixture to dryness; and (5) analysing the sample.

Glossary
2-CT: 2-ch lorotrityl
ACN: acetonitrile
aq: aqueous
Cupral: diethylcarbamodithioic acid, sodium salt
CV: column volume
DCM: dichloromethane
DIPEA: diisopropylethylamine
DIPO: diisopropyl oxide
DMF: N,N-dimethylformamide
Fmoc: fluorenylmethoxycarbonyl
HBTU: (2-(1H-benzotriazol-1-yl)-1,1,3,3-tetramethyluronium hexafluorophosphate
HFIP: 1,1,1,3,3,3-hexafluoroisopropanol
HPLC: high performance liquid chromatography
LCMS: liquid chromatography-mass spectrometry
LCUV: liquid chromatography-ultraviolet
NMR: nuclear magnetic resonance
Pd Tetrakis: tetrakis(triphenylphosphine)palladium(0)
PFA: paraformaldehyde
TEA: triethylamine
TFA: trifluoroacetic acid
THF: tetrahydrofuran
TIPS: triisopropyl silane
TLC: thin layer chromatography

### Example 1—Synthesis of HYNIC-iPSMA

HYNIC-iPSMA was prepared by the methods described herein according to Scheme 1. Specific reaction conditions for Scheme 1 are provided below, although any suitable conditions known in the art for the relevant transformation of steps 1-5 may be used.

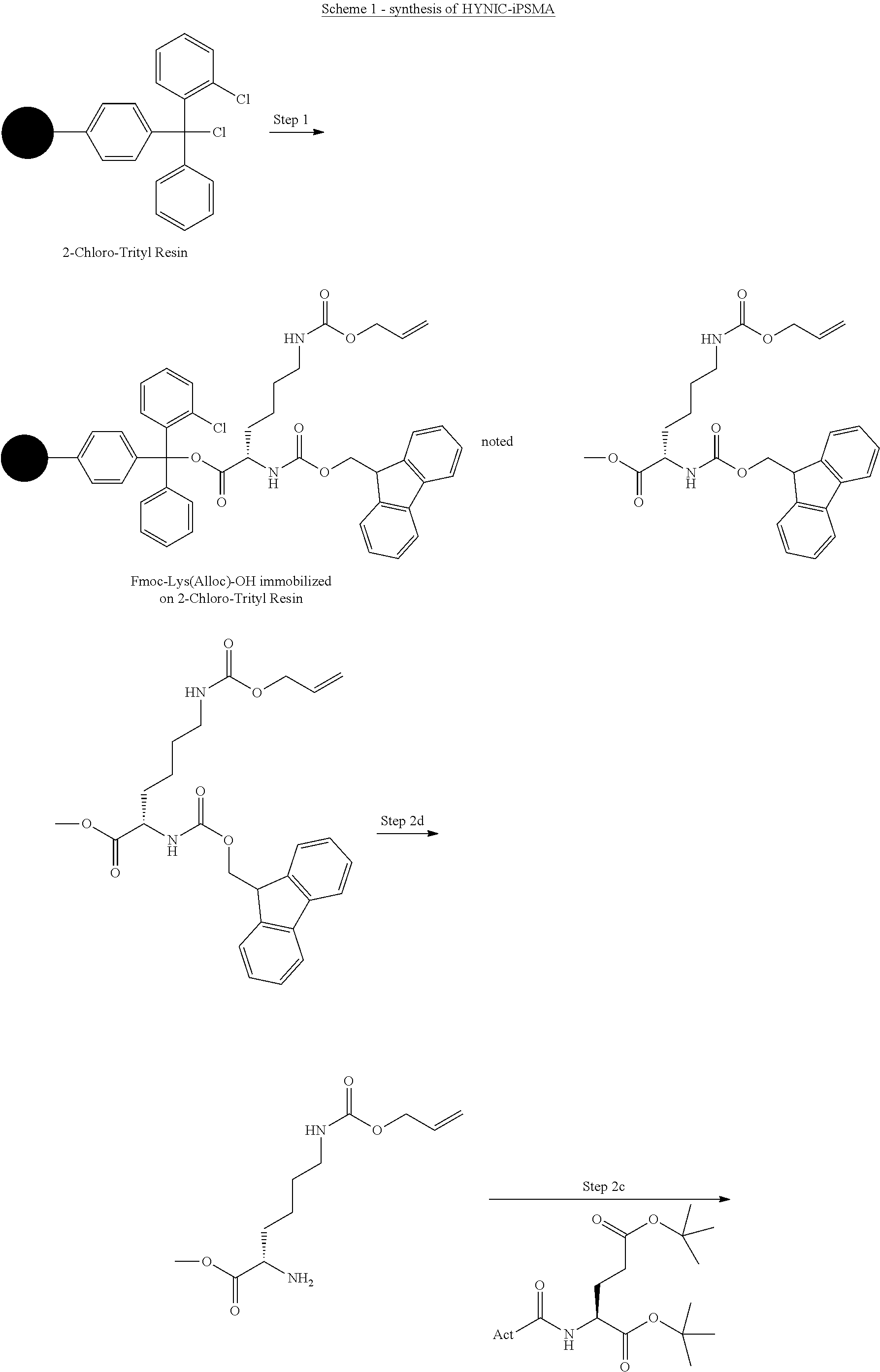

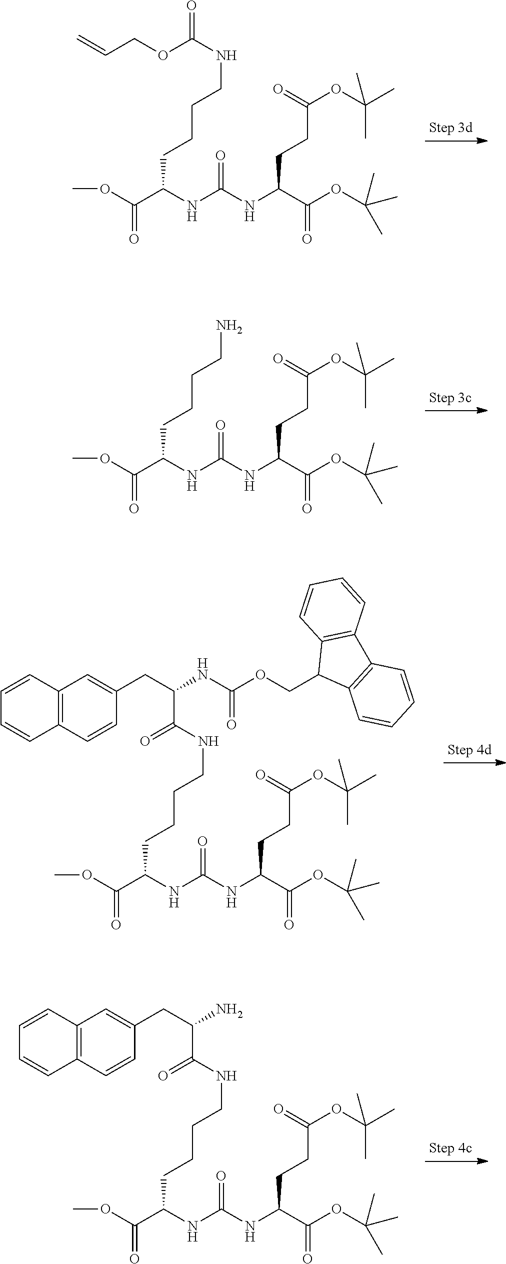

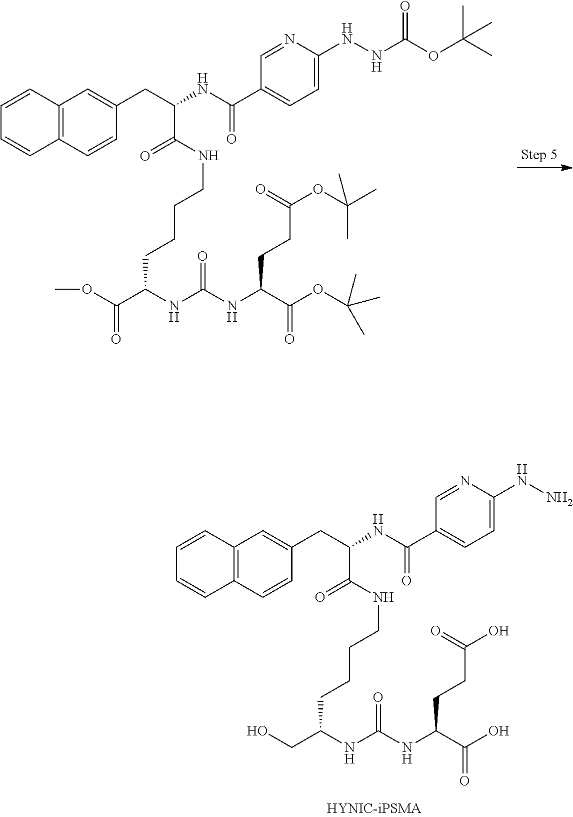 [SMILES: COC(=O)[C@H](CCCCNC(=O)[C@H](Cc1ccc2ccccc2c1)NC(=O)c1ccc(NNC(=O)OC(C)(C)C)nc1)NC(=O)N[C@@H](CCC(=O)OC(C)(C)C)C(=O)OC(C)(C)C ; NNc1ccc(C(=O)N[C@@H](Cc2ccc3ccccc3c2)C(=O)NCCCC[C@@H](CO)NC(=O)N[C@@H](CCC(=O)O)C(=O)O)cn1]

Step 1—Loading Fmoc-Lys(Alloc)-OH on 2-chlorotrityl Resin

Step 1 involved immobilisation of Fmoc-Lys(Alloc)-OH onto 2-chlorotrityl (2-CT) resin using DIPEA and DCM. DIPEA was used in excess to avoid resin degradation under acidic conditions. Unreacted starting material and DIPEA were removed with DCM washing.

Procedure: 2-CT resin was mixed with DCM (13-16V) and stirred using N₂ bubbling at room temperature. A solution of Fmoc-Lys(Alloc)-OH (0.95-0.97 eq) and DIPEA (4.2 eq) in DCM (3V) was added. The mixture was stirred using N₂ bubbling at room temperature for at least 3 hours. The mixture was filtered. The resin was washed four times with DCM (4×3V) for 10 minutes, four times with a DCM/MeOH/DIPEA (17:2:1) mixture (4×7V) for 10 minutes, then four times with DCM (4×3V) for 10 minutes. The resin was then dried on the filter overnight at room temperature. The product was analysed by HPLC.

The effect of reaction duration on yield (loading of Fmoc-Lys(Alloc)-OH onto 2-CT resin) was investigated. At a small scale of 1g resin and 0.619g (1.37 mmol) Fmoc-Lys(Alloc)-OH, reaction durations of 3, 6 and 16 hours achieved loading of at least 99%. At a large scale of 45 g resin and 27.84 g (62.93 mmol) Fmoc-Lys(Alloc)-OH, a reaction duration of 3 hours achieved 72% loading.

Step 2—Fmoc Deprotection and H-Glu(O-tert-butyl)-O-tert-butyl Coupling

Step 2 involved deprotection of Fmoc using DMF and piperidine (step 2d), followed by coupling with activated H-Glu(O-tert-butyl)-O-tert-butyl (Act=1H-imidazole) using DIPEA and DMF (step 2c).

Procedure—step 2d: The resin was washed 4 times with DMF (4×5V). The resin was mixed with DMF/piperidine mixture (1:1, 10V) using N₂ bubbling at room temperature for 15 minutes to 1 hour, and then filtered. This step was repeated 5-6 times. The resin was then washed four times with DMF (4×5V) and four times with DCM (4×5V). The resin was subsequently dried on the filter overnight at room temperature. LCMS analysis of the cleaved resin confirmed formation of the product. No starting material was detected in the analysis.

Procedure—step 2c: The resin was washed once with DMF (10V). The resin was mixed with DMF (5V) and stirred using N₂ bubbling at room temperature. A solution of activated H-Glu(O-tert-butyl)-O-tert-butyl (3.2 eq) and DIPEA (1.3 eq) in DMF (12V) was added. The mixture was stirred using N₂ bubbling at room temperature for at least 3 hours, typically at least 16 hours. The mixture was filtered. The resin was washed four times with DMF (4×10V), then four times with DCM (4×10V). The resin was then dried on the filter overnight at room temperature. LCMS and HPLC analysis of the cleaved resin confirmed formation of the product.

Step 3—Alloc Deprotection and Fmoc-2-Nal-OH Coupling

Step 3 involves deprotection of Alloc using Pd Tetrakis, morpholine and DCM (step 3d), followed by coupling with Fmoc-2-Nal-OH using HBTU and DIPEA in DMF (step 3c).

Procedure—step 3d: The resin was mixed with DCM (18V) and stirred using N₂ bubbling at room temperature. Morpholine (60 eq) was added, then Pd Tetrakis (0.1-1.0 eq, typically 0.3 eq) was subsequently added. The mixture was stirred for at least 2 hours using N₂ bubbling at room temperature and the reaction vessel protected from light. The mixture was then filtered. The resin was washed four times with DCM (4×5V), four times with DMF (4×5V), ten times with a solution of DIPEA (1%) in DMF (10×5V), ten times with a solution of Cupral (15 mg/mL in DMF) (10×5V), four times with DMF (4×5V), and four times with DCM (4×5V). The resin was subsequently dried on the filter overnight at room temperature. HPLC analysis of the cleaved resin confirmed formation of the product.

Procedure—step 3c: The resin was washed once with DMF (10V). The resin was mixed with DMF (5V) using N₂ bubbling at room temperature. A solution of Fmoc-2-Nal-OH (4.0 eq), HBTU (3.96 eq) and DIPEA (4.0 eq) in DMF (10V) was added. The mixture was stirred using N₂ bubbling at room temperature for at least 6 hours. The resin was washed four times with DMF (4×10V), then four times with DCM (4×10V). The resin was then dried on the filter overnight at room temperature. HPLC analysis of the cleaved resin confirmed formation of the product.

Step 4—Fmoc Deprotection and Boc-Protected HYNIC Coupling

Step 4 involves deprotection of Fmoc using DMF and piperidine (step 4d), followed by coupling with Boc-protected HYNIC using HBTU and DIPEA in DMF (step 4c).

Procedure—step 4d: The resin was washed 4 times with DMF (4×5V) at room temperature using N₂ bubbling for at least 10 minutes. The resin was mixed with DMF/piperidine mixture (1:1, 10V) using N₂ bubbling at room temperature for at least 15 minutes, and then filtered. This step was repeated 4 times. The resin was then washed four times with DMF (4×5V) at room temperature using N₂ bubbling for 10 minutes. HPLC analysis of the cleaved resin confirmed formation of the product.

Procedure—step 4c: The resin was mixed with DMF (5V) using N₂ bubbling at room temperature. A solution of Boc-HYNIC (4.0 eq), HBTU (3.96 eq) and DIPEA (4.0 eq) in DMF (10V) was added. The mixture was stirred using N₂ bubbling at room temperature for at least 6 hours. The resin was washed four times with DMF (4×10V), then four times with DCM (4×10V). The resin was then dried for at least 12 hours at room temperature. HPLC analysis of the cleaved resin confirmed formation of the product.

Step 5—Resin Cleavage and Deprotection

The resin was mixed in a flask in a TFA/TIPS/H₂O solution (17:22.5:0.5, 17V) at room temperature for 2-3 hours. The mixture was filtered and the solid washed two times with acetonitrile (2×5V). The filtrate was concentrated under reduced pressure and residual TFA was co-evaporated two times with acetonitrile (2×5V). The residue was then solubilised in water (10V) and acetonitrile (2.5V) and lyophilised to provide a solid.

The solid was purified by C18 chromatography using the following conditions:
Conditioning: Loading<2.5%, typically 1.2-2.4%
   H₂O/ACN (0.1% TFA) (90:10):10CV
   H₂O/ACN (0.1% TFA) (90:10→70:30):30CV
   H₂O/ACN (0.1% TFA) (70:30→0:100):5CV
   H₂O/ACN (0.1% TFA) (0:100):5CV
Solubilisation of the solid: 6.5V of H₂O/ACN (8:2)

Fractions containing the product were combined and lyophilised to provide a solid. The solid was solublised in water (18.6V) and acetonitrile (1.4V) and then lyophilised to provide HYNIC-iPSMA. The purified product was analysed by HPLC and NMR.

### Example 2—Synthesis of HYNIC-iPSMA

HYNIC-iPSMA may also be prepared by the methods described herein according to Scheme 2. In Scheme 2, any suitable conditions known in the art for the relevant transformation of steps 1-6d may be used.

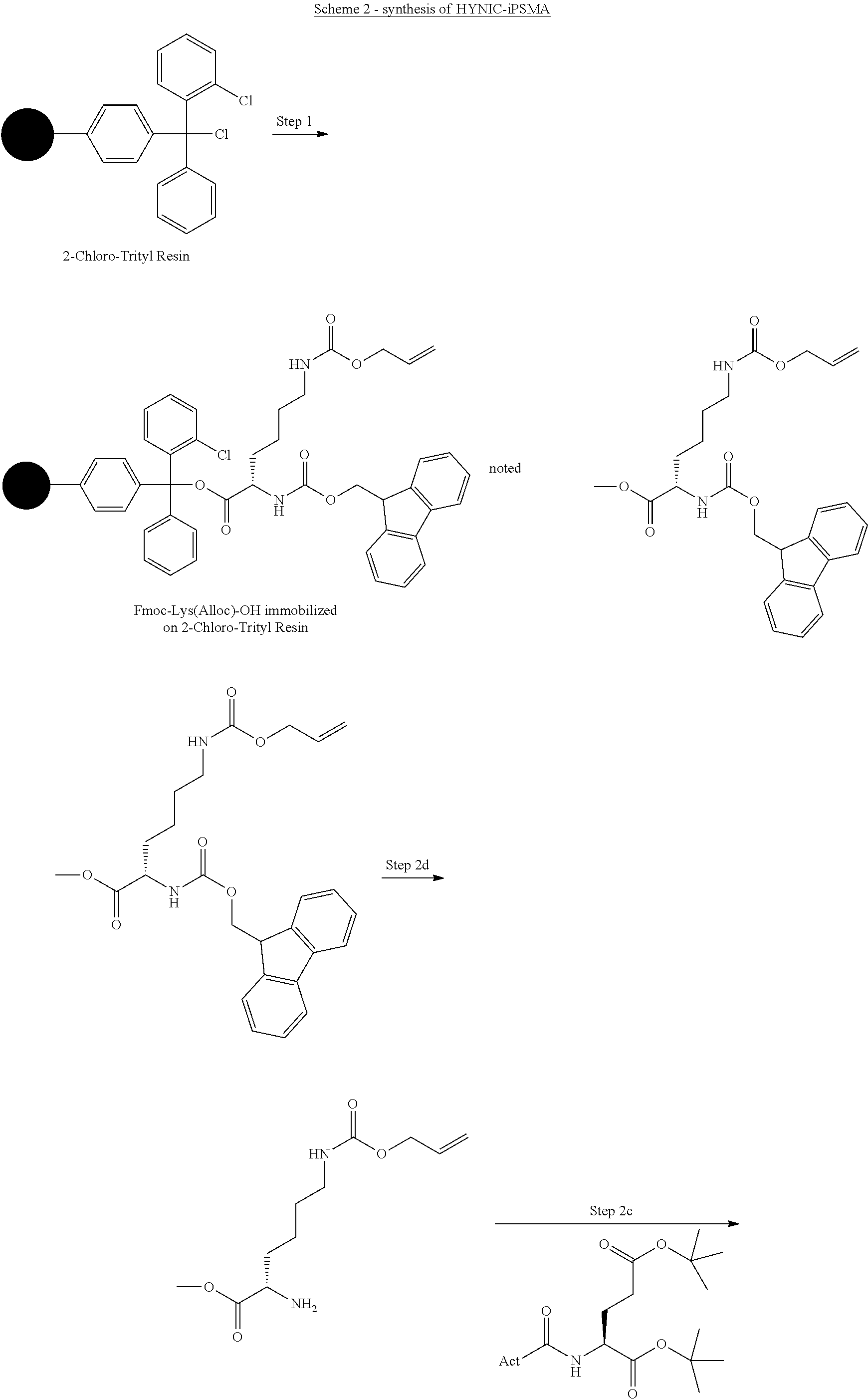

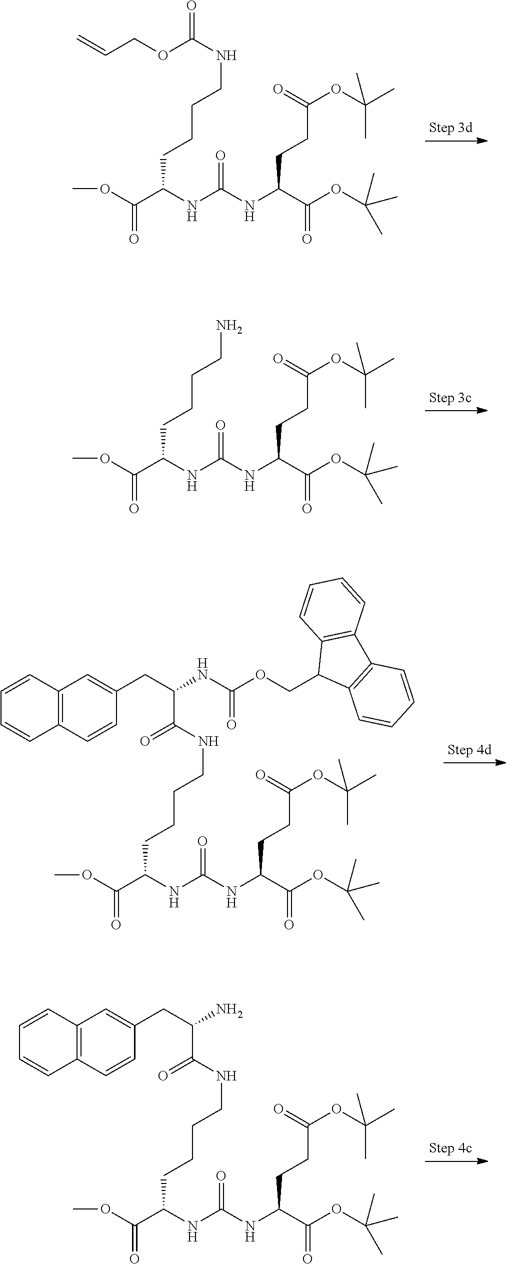

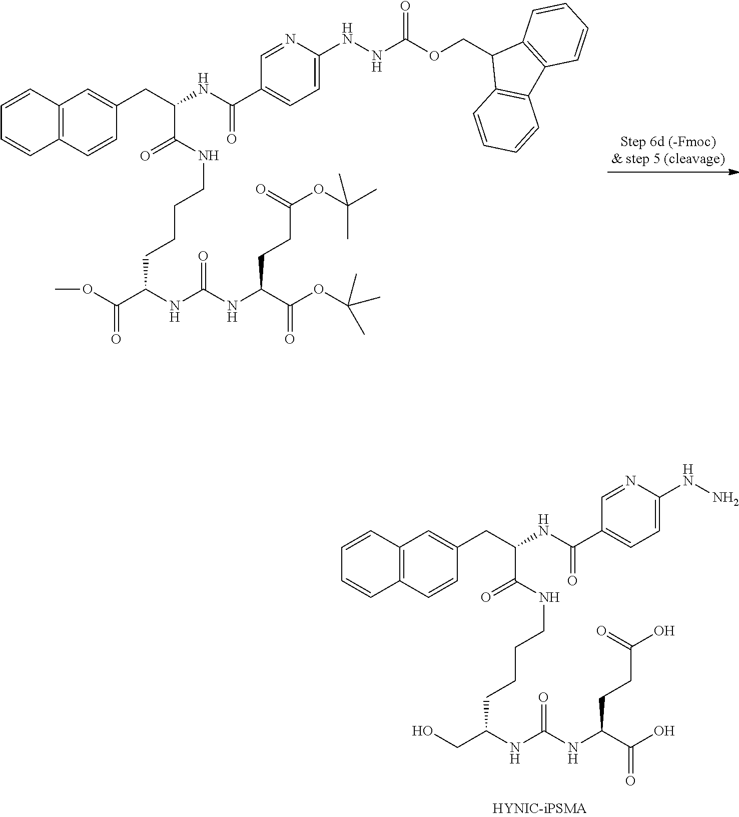 [SMILES: COC(=O)[C@H](CCCCNC(=O)[C@H](Cc1ccc2ccccc2c1)NC(=O)c1ccc(NNC(=O)OCC2c3ccccc3-c3ccccc32)nc1)NC(=O)N[C@@H](CCC(=O)OC(C)(C)C)C(=O)OC(C)(C)C ; NNc1ccc(C(=O)N[C@@H](Cc2ccc3ccccc3c2)C(=O)NCCCC[C@@H](CO)NC(=O)N[C@@H](CCC(=O)O)C(=O)O)cn1]

### Example 3—Synthesis of DOTA-HYNIC-iPSMA

DOTA-HYNIC-iPSMA was prepared by the methods described herein according to Scheme 3. Specific reaction conditions for Scheme 3 are provided below, although any suitable conditions known in the art for the relevant transformation of steps 1-7 may be used.

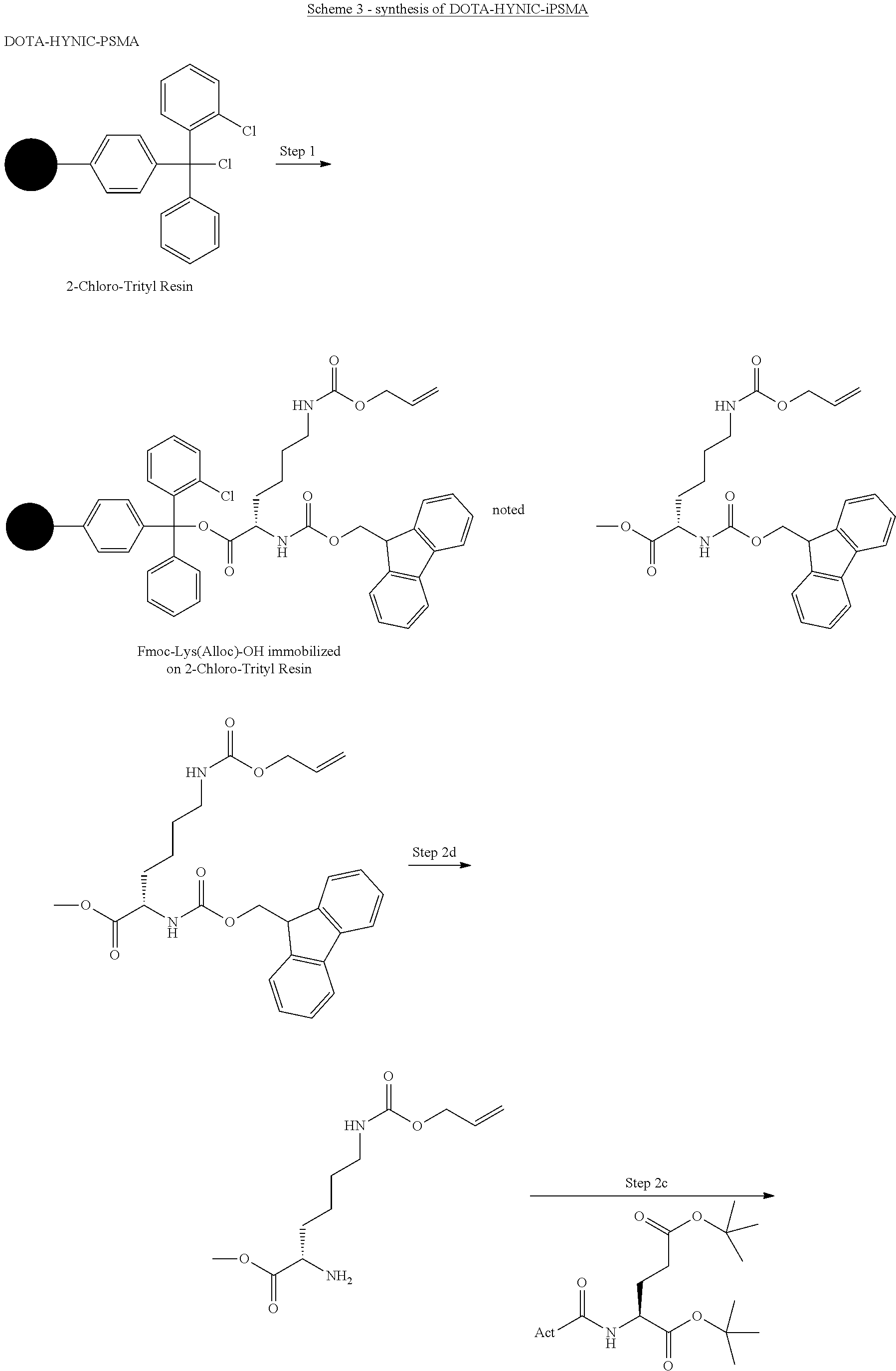

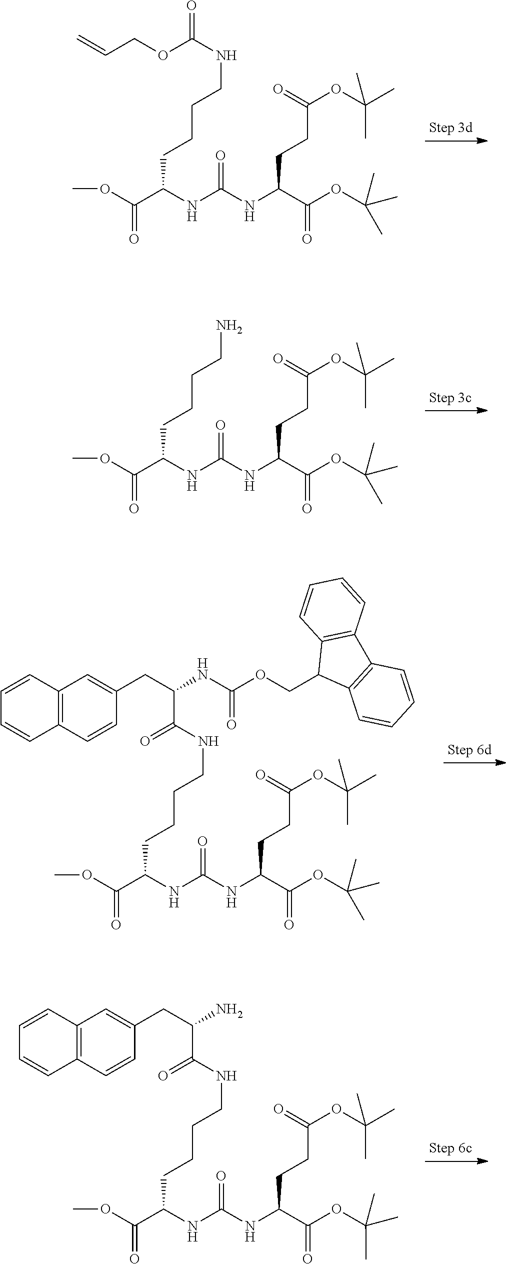

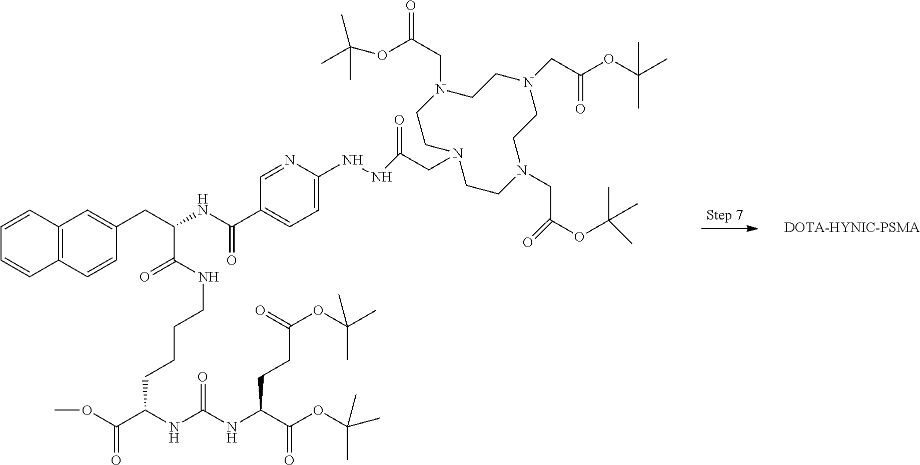 [SMILES: COC(=O)[C@H](CCCCNC(=O)[C@H](Cc1ccc2ccccc2c1)NC(=O)c1ccc(NNC(=O)CN2CCN(CC(=O)OC(C)(C)C)CCN(CC(=O)OC(C)(C)C)CCN(CC(=O)OC(C)(C)C)CC2)nc1)NC(=O)N[C@@H](CCC(=O)OC(C)(C)C)C(=O)OC(C)(C)C]

Steps 1-3 of Scheme 3 were performed following the procedures in Example 1.

Step 6—Fmoc Deprotection and DOTA-HYNIC Coupling

Step 6 involves deprotection of Fmoc using DMF and piperidine (step 6d), followed by coupling with DOTA-HYNIC using HBTU and DIPEA in DMF (step 6c). The synthesis of DOTA-HYNIC is provided in Example 6.

Procedure—step 6d: The resin was washed 4 times with DMF (4×5V) at room temperature using N₂ bubbling for 10 minutes. The resin was mixed with DMF/piperidine mixture (1:1, 10V) using N₂ bubbling at room temperature for at least 15 minutes, and then filtered. This step was repeated at least 5 times. The resin was then washed four times with DMF (4×5V) at room temperature using N₂ bubbling for 10 minutes. The resin was dried on the filter overnight at room temperature. HPLC analysis of the cleaved resin confirmed formation of the product.

Procedure—step 6c: The resin was washed once with DMF (10V). The resin was mixed with DMF (5V) using N₂ bubbling at room temperature. A solution of DOTA-HYNIC (2.0-3.0 eq), HBTU (ratio equivalent of HBTU/equivalents of DOTA-HYNIC 0.90-0.98) and DIPEA (eq of DIPEA=eq of DOTA-HYNIC+1) in DMF (10V) was added. The mixture was stirred using N₂ bubbling at room temperature for at least 6 hours. The resin was washed four times with DMF (4×10V), then four times with DCM (4×10V). The resin was then dried on the filter overnight at room temperature. HPLC analysis of the cleaved resin confirmed formation of the product.

Step 7—Resin Cleavage and Deprotection

The resin was mixed in a flask in a TFA/TIPS/H₂O solution (19:2.5:2.5, 10V) at room temperature for 5-7 hours. The mixture was filtered and the solid washes two times with acetonitrile (2×5V). The filtrate was concentrated under reduced pressure and residual TFA was co-evaporated two times with acetonitrile (2×5V). The residue was then solubilised in water (9V) and acetonitrile (1V) and lyophillised to provide a solid.

The solid was purified by C18 chromatography using the following conditions:
Column size: 25 g for 300-600 mg crude loaded; 800 g for 17.3 g crude loaded
Conditioning: Loading 1.2-2.4 w/w %
Solubilisation of the solid: H₂O/ACN (0.1% TFA)
Elution flowrate: 32 mL/min for 25 g column; 160 mL/min for 800 g column

Fractions containing the product were combined and lyophilised to provide a solid. The solid was solubilised in water (18V) and acetonitrile (2V) and then lyophilised to provide DOTA-HYNIC-PSMA. Alternatively, the solid was twice solubilised in water (20V) and then lyophilised to provide DOTA-HYNIC-PSMA. Formation of DOTA-HYNIC-PSMA was confirmed by ¹H NMR and ¹³C NMR.

### Example 4—Synthesis of DOTA-HYNIC-iPSMA

DOTA-HYNIC-iPSMA may also be prepared by the methods described herein according to Scheme 4. In Scheme 4, any suitable conditions known in the art for the relevant transformation of steps 1-7 may be used.

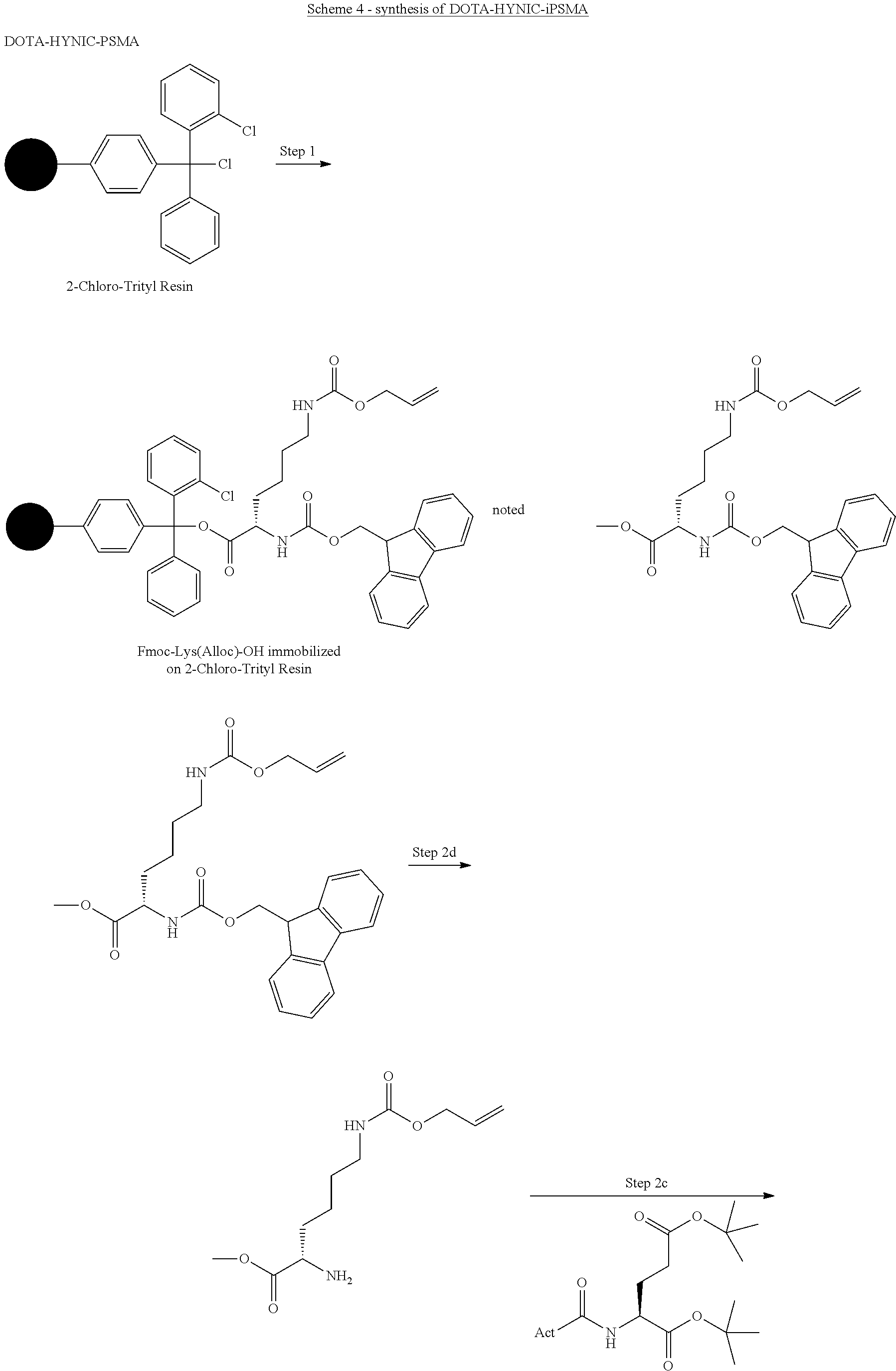

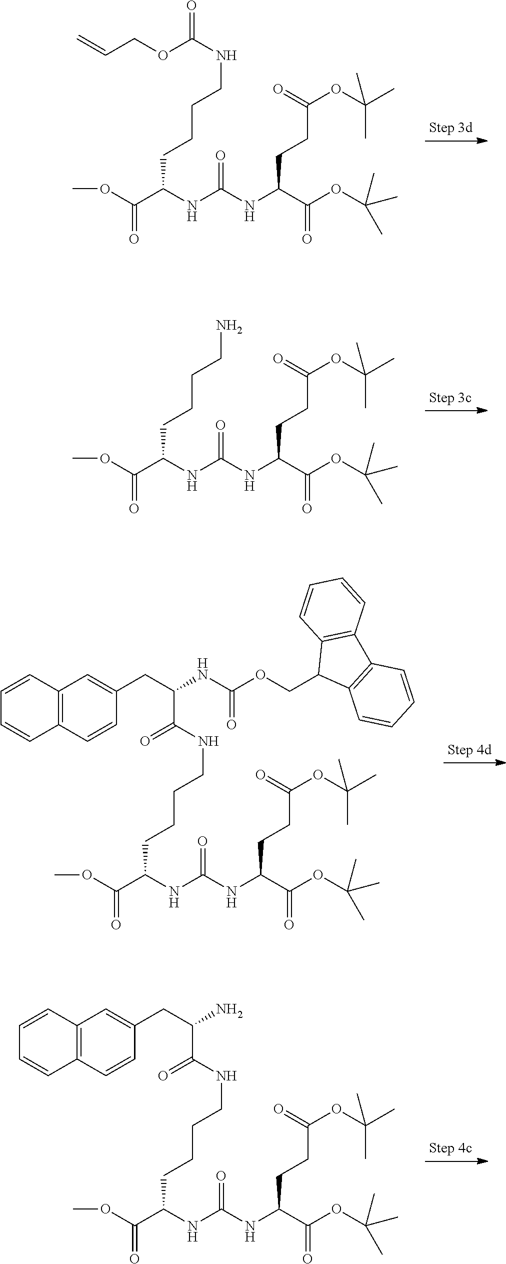

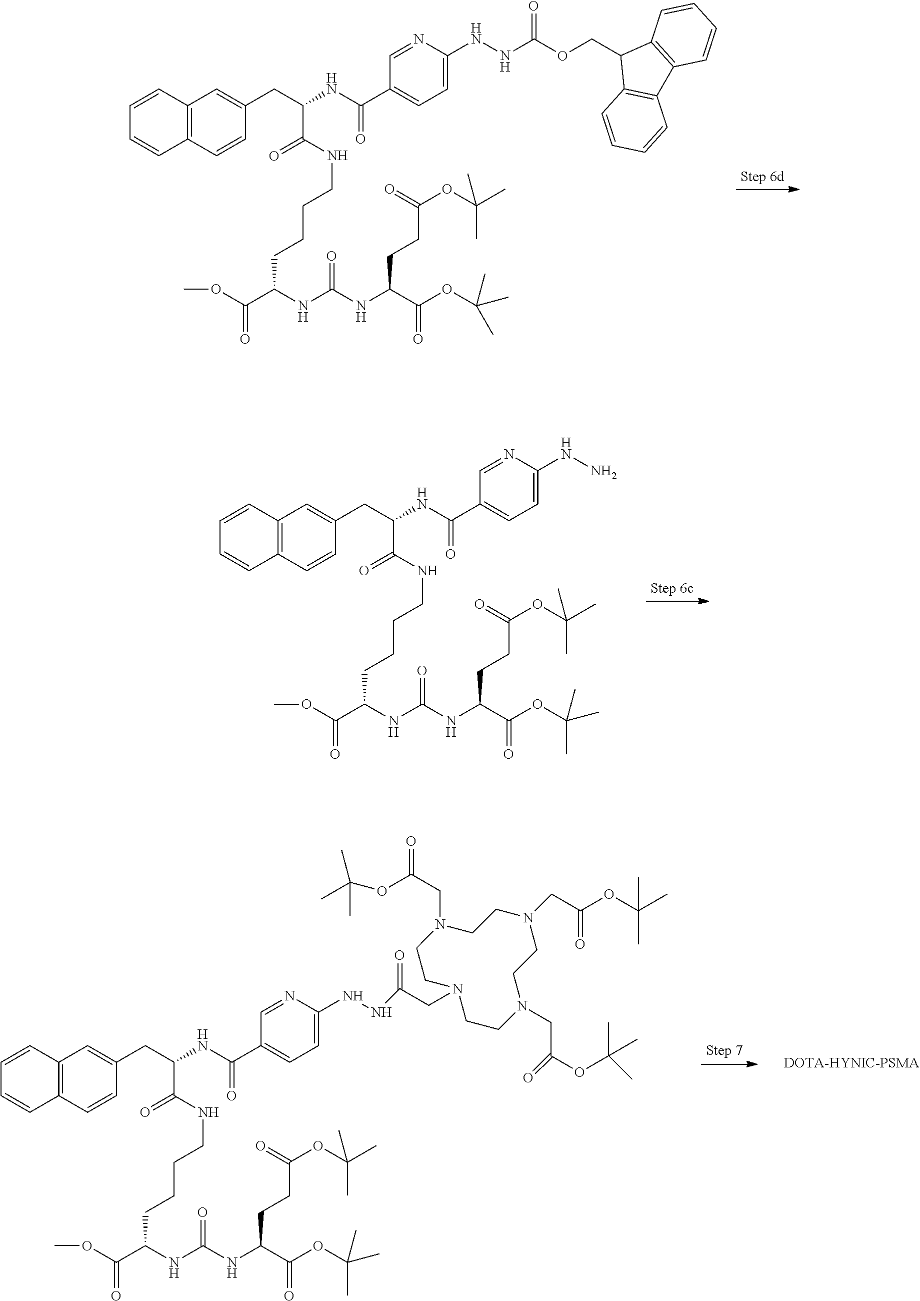 [SMILES: COC(=O)[C@H](CCCCNC(=O)[C@H](Cc1ccc2ccccc2c1)NC(=O)c1ccc(NNC(=O)OCC2c3ccccc3-c3ccccc32)nc1)NC(=O)N[C@@H](CCC(=O)OC(C)(C)C)C(=O)OC(C)(C)C ; COC(=O)[C@H](CCCCNC(=O)[C@H](Cc1ccc2ccccc2c1)NC(=O)c1ccc(NN)nc1)NC(=O)N[C@@H](CCC(=O)OC(C)(C)C)C(=O)OC(C)(C)C ; COC(=O)[C@H](CCCCNC(=O)[C@H](Cc1ccc2ccccc2c1)NC(=O)c1ccc(NNC(=O)CN2CCN(CC(=O)OC(C)(C)C)CCN(CC(=O)OC(C)(C)C)CCN(CC(=O)OC(C)(C)C)CC2)nc1)NC(=O)N[C@@H](CCC(=O)OC(C)(C)C)C(=O)OC(C)(C)C]

### Example 5—Synthesis of PSMA-11

PSMA-11 is prepared by the methods described herein according to Scheme 5. Specific reaction conditions for Scheme 5 are provided below, although any suitable conditions known in the art for the relevant transformation of steps 1-10 may be used.

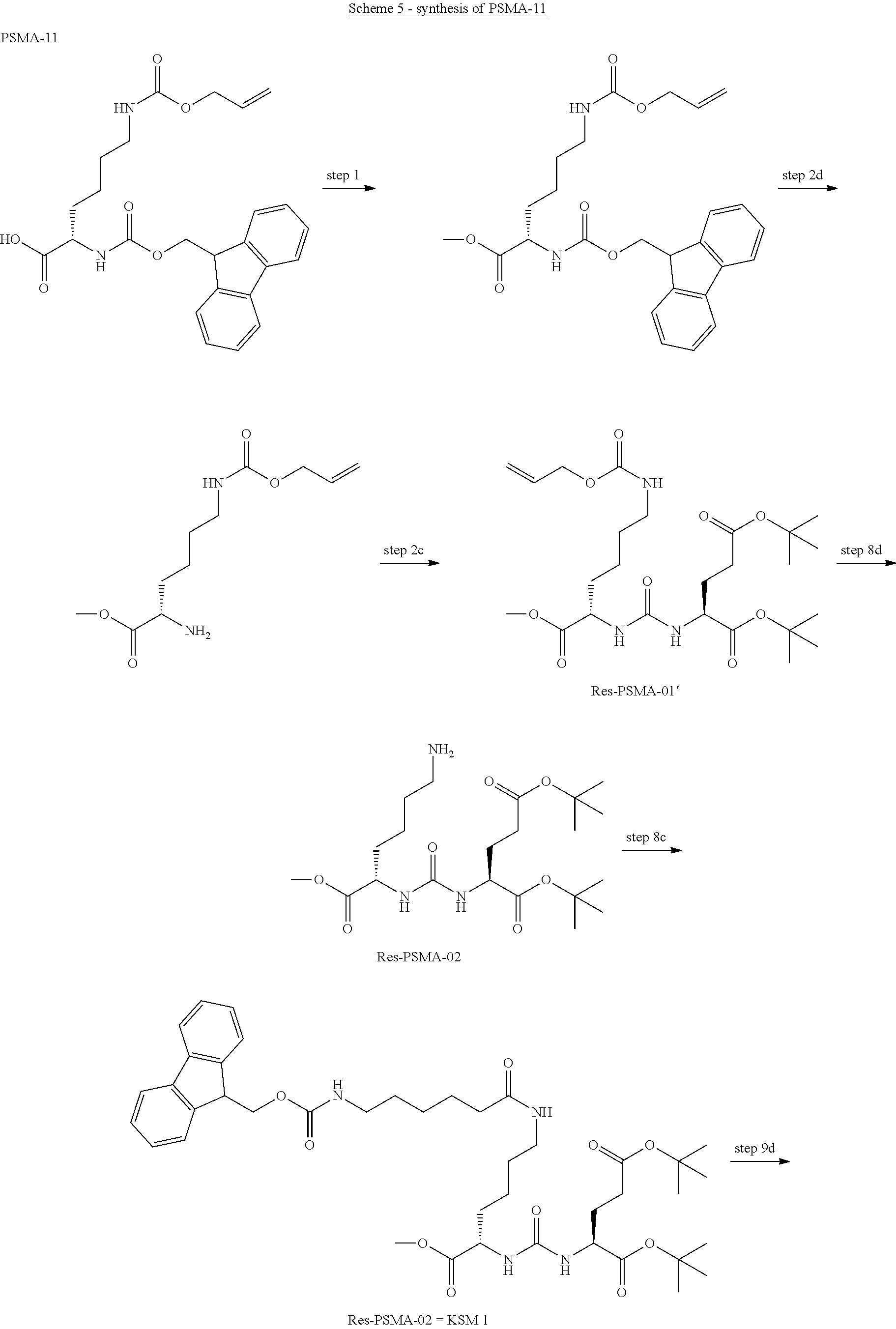

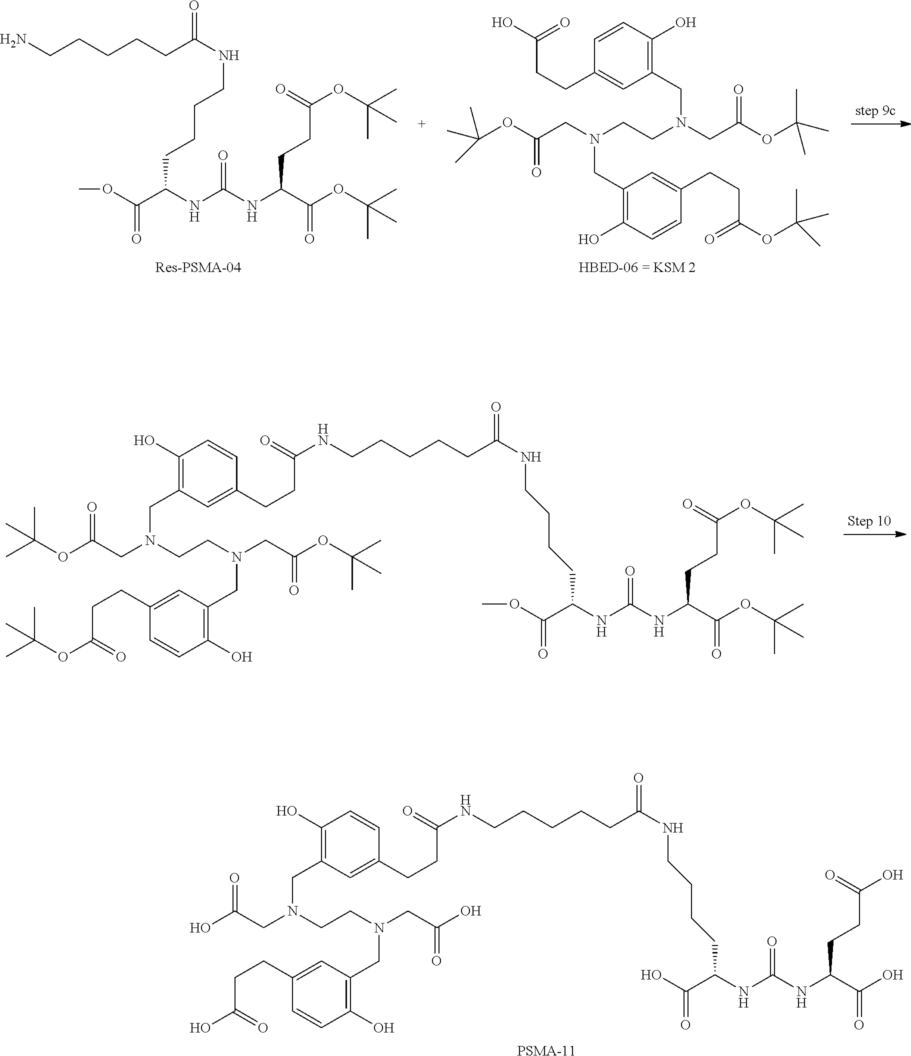

Steps 1 and 2 of Scheme 5 were performed following the procedures in Example 1.

Step 8—Alloc Deprotection and Fmoc-6-AHA-OH Coupling

Step 8 involved deprotection of Alloc using Pd Tetrakis, morpholine and DCM (step 8d), followed by coupling with Fmoc-6-AHA-OH using HBTU and DIPEA in DMF (step 8c).

Procedure—step 8d: The resin was mixed with DCM (18V) using N₂ bubbling at room temperature. Morpholine (60 eq) was added, then Pd Tetrakis (0.3 eq) was subsequently added. The mixture was stirred for at least 2 hours using N₂ bubbling at room temperature and the reaction vessel protected from light. The mixture was then filtered. The resin was washed four times four times with DCM (4×5V), four times with DMF (4×5V), ten times with a solution of DIPEA (1%) in DMF (10×5V), ten times with a solution of Cupral (15 mg/mL in DMF) (10×5V), four times with DMF (4×5V), and four times with DCM (4×5V). The resin was subsequently dried on the filter overnight at room temperature. HPLC analysis of the cleaved resin confirmed formation of the product.

Procedure—step 8c: The resin was washed once with DMF (10V). The resin was mixed with DMF (5V) using N₂ bubbling at room temperature. A solution of Fmoc-6-AHA-OH (4.0 eq), HBTU (3.96 eq) and DIPEA (4.0 eq) in DMF (10V) was added. The mixture was stirred using N₂ bubbling at room temperature for 6 hours. The resin was washed four times with DMF (4×10V), then four times with DCM (4×10V). The resin was then dried on the filter overnight at room temperature. LCUV analysis of the cleaved resin confirmed formation of the product. The cleaved product was also analysed by ¹H and ¹³C NMR.

Step 9—Fmoc Deprotection and HBED-06 Coupling

Step 9 involves deprotection of Fmoc using DMF and piperidine (step 9d), followed by coupling with HBED-06 using HBTU and DIPEA in DMF (step 9c). The synthesis of HBED-06 is provided in Example 7. HBED-06 may alternatively be prepared by methods known in the art, for example as described in Makarem et al (Synlett 2018, 29, 1239-1243), the entirety of which is incorporated herein by this cross-reference.

Procedure—step 9d: The resin was washed 4 times with DMF (4×5V) at room temperature using N₂ bubbling for 10 minutes. The resin was mixed with DMF/piperidine mixture (1:1, 10V) using N₂ bubbling at room temperature for 15 minutes, and then filtered. This step was repeated 5 times. The resin was then washed four times with DMF (4×5V) at room temperature using N₂ bubbling for 10 minutes. Analysis of the cleaved resin confirmed formation of the product.

If the coupling step (step 9c) is performed more than 2-3 days after the deprotection step, the following steps are further conducted to avoid degradation during intermediary storage. The resin is washed four times with DCM (4×5V) at room temperature using N₂ bubbling for 10 minutes. The resin is then dried under reduced pressure until no mass variation. At the beginning of the coupling step, a supplementary wash with DMF (10V) at room temperature using N₂ bubbling should be performed to remove residual DCM.

Procedure—step 9c: The resin was mixed with DMF (5V) and stirred using N₂ bubbling at room temperature. A solution of HBED-06 (4.0 eq), HBTU (3.96 eq) and DIPEA (4.0 eq) in DMF (10V) was added. The mixture was stirred using N₂ bubbling at room temperature for 6 hours. The resin was washed four times with DMF (4*10V), then four times with DCM (4×10V). The resin was then dried under vacuum until no mass variation. Analysis of the cleaved resin confirmed formation of the product.

Step 10—Resin Cleavage and Boc Deprotection

The resin was mixed in a flask in a TFA/TIPS/H₂O solution (17:0.5:0.5, 18V) at room temperature for 2 hours. The mixture was filtered and the solid washed two times with acetonitrile (10V). The filtrate was concentrated under reduced pressure and residual TFA was co-evaporated two times with acetonitrile (2×5V). The residue was then solubilised in water and lyophilised to provide a pale yellow solid.

The solid was purified by C18 chromatography using the following conditions:
Column: AIT C18 40-60 μm
Conditioning: Loading 0.7%
   H₂O/ACN (70:30):2CV
   H₂O/ACN (0.1% TFA) (70:30→90:10):3CV
   H₂O/ACN (0.1% TFA) (90:10):3CV
Solubilisation of the solid: 6V of H₂O/ACN (5:1) (0.1% TFA)
Elution: H₂O/ACN (0.1% TFA) (90:10 4 80:20):23CV

Fractions containing the product were combined and lyophilised to provide PSMA-11.

### Example 6—Synthesis of DOTA-HYNIC Intermediate

DOTA-HYNIC was prepared by the method according to Scheme 6.

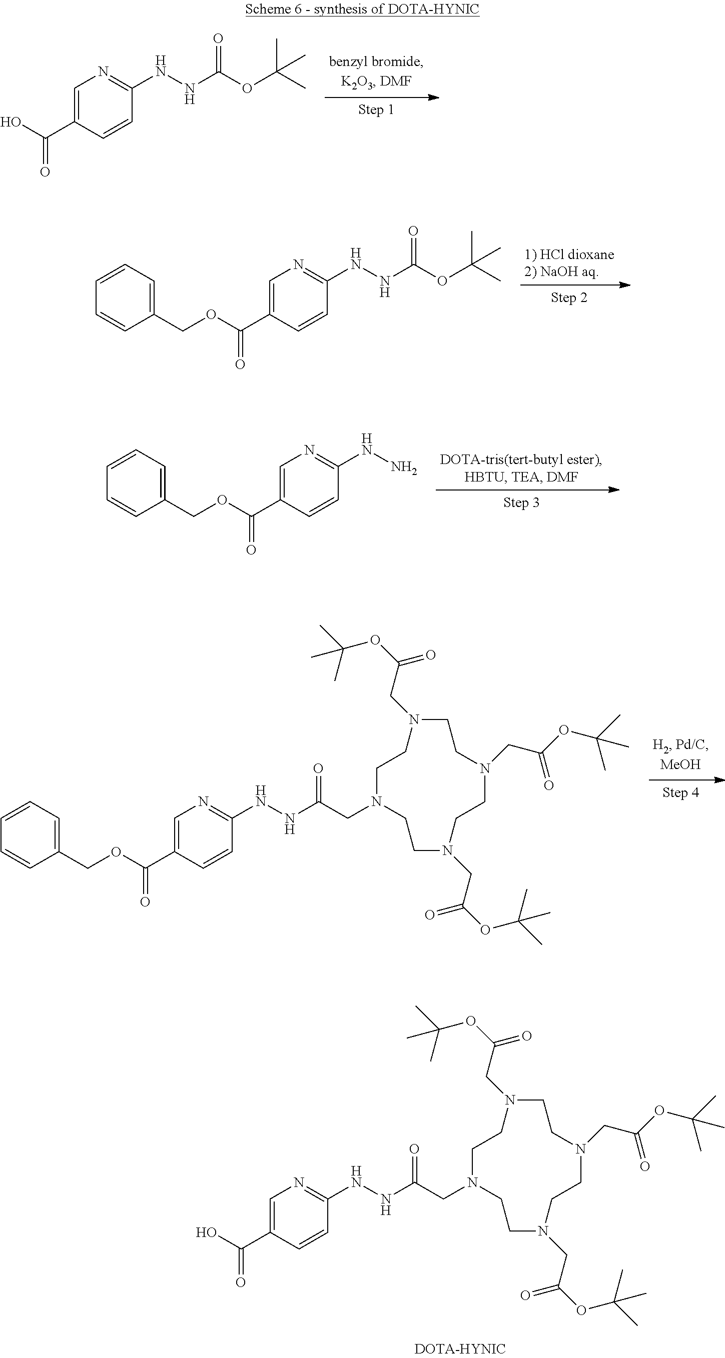 [SMILES: CC(C)(C)OC(=O)NNc1ccc(C(=O)O)cn1 ; CC(C)(C)OC(=O)NNc1ccc(C(=O)OCc2ccccc2)cn1 ; NNc1ccc(C(=O)OCc2ccccc2)cn1 ; CC(C)(C)OC(=O)CN1CCN(CC(=O)NNc2ccc(C(=O)OCc3ccccc3)cn2)CCN(CC(=O)OC(C)(C)C)CCN(CC(=O)OC(C)(C)C)CC1 ; CC(C)(C)OC(=O)CN1CCN(CC(=O)NNc2ccc(C(=O)O)cn2)CCN(CC(=O)OC(C)(C)C)CCN(CC(=O)OC(C)(C)C)CC1]

Step 1—Benzyl Protection

Boc-HYNIC and K₂CO₃ (1.1 eq) were mixed in DMF (10V) under nitrogen atmosphere and benzyl bromide (BnBr; 1.1eq) was added dropwise, at room temperature. The mixture was stirred overnight at room temperature then, ethyl acetate (30V) was added and the resulting mixture was washed three times with water (3×15V). The recombined aqueous layers were extracted with ethyl acetate (15V). Then, the recombined organic layers were washed again two times with water (2×15V) then with brine (7.5V) before being concentrated under reduced pressure.

The crude product was purified using silica chromatography:
Conditioning: Loading 6.1%
DCM/EtOAc (100:0):2CV
DCM/EtOAc (95:5):2CV
DCM/EtOAc (90:10):8CV
DCM/EtOAc (80:20):4CV
DCM/EtOAc (75:25):4CV
DCM/EtOAc (70:30):8CV

¹H NMR analysis of the obtained solid confirmed formation of the fully protected product.

Step 2—Boc Deprotection

The benzyl ester protected intermediate was solubilised in dioxane (10V) under nitrogen atmosphere and a solution of HCl 4N in dioxane (1.1 eq of HCl) was added by portions at room temperature. The reaction mixture was stirred at room temperature overnight. Then, the mixture was cooled at 0° C. using ice bath and neutralized using NaOH 6N without exceeding a temperature of 20° C. The resulting solution was diluted with water (7V) and extracted three times with ethyl acetate (3×7V). The recombined organic layers were washed with brine (7V) and concentrated under reduced pressure.

The crude product was purified using silica chromatography:
Conditioning: Loading 8.3%
DCM/ACN (+1% of NH₃ (7N) in MeOH) (90:10):1CV
DCM/ACN (+1% of NH₃ (7N) in MeOH) (80:20):1CV
DCM/ACN (+1% of NH₃ (7N) in MeOH) (70:30):4CV
DCM/ACN (+1% of NH₃ (7N) in MeOH) (50:50):4CV
DCM/ACN (+1% of NH₃ (7N) in MeOH) (30:70):4CV
DCM/ACN (+1% of NH₃ (7N) in MeOH) (10:90):4CV

¹H NMR analysis of the obtained solid confirmed formation of the free hydrazine product.

Step 3—DOTA Coupling

The hydrazine starting product (3.0 eq) and trisiBu-DOTA were solubilized in DMF (10V) under nitrogen atmosphere and trimethylamine (2.0 eq) then HBTU (1.2 eq) were subsequently added to the mixture at room temperature. The reaction mixture was stirred at room temperature for 4 hours then diluted with ethyl acetate (50V). The organic layer was washed two times with a saturated solution of NaHCO₃ (2×15 V), two times with water (2×15V) then with brine (15V) before being concentrated under reduced pressure.

The crude product was purified using silica chromatography:
Conditioning: Loading 5.3%
DCM/ACN (100:0):1CV
DCM/ACN (98:2):2CV
DCM/ACN (95:5):4CV
DCM/ACN (90:10):4CV
DCM/ACN (85:15):6CV
DCM/ACN (80:20):4CV

¹H NMR analysis of the obtained solid confirmed formation of the DOTA-functionalised product.

Step 4—Benzyl Ester Deprotection

The DOTA-functionalised product was solubilised in MeOH (10V) under nitrogen atmosphere and wet palladium on carbon was added to the mixture before purging the atmosphere with hydrogen. The reaction mixture was stirred under hydrogen atmosphere for 3 hours at room temperature then was filtered on a celite pad. The cake was washed with MeOH (4×15V) and the filtrate was concentrated under reduced pressure. ¹H NMR analysis of the obtained solid confirmed formation of DOTA-HYNIC.

### Example 7—Synthesis of HBED-06 Intermediate

HBED-06 was prepared by the method according to Scheme 7. Specific reaction conditions for the final step in the synthesis (saponification with LiOH) is provided below.

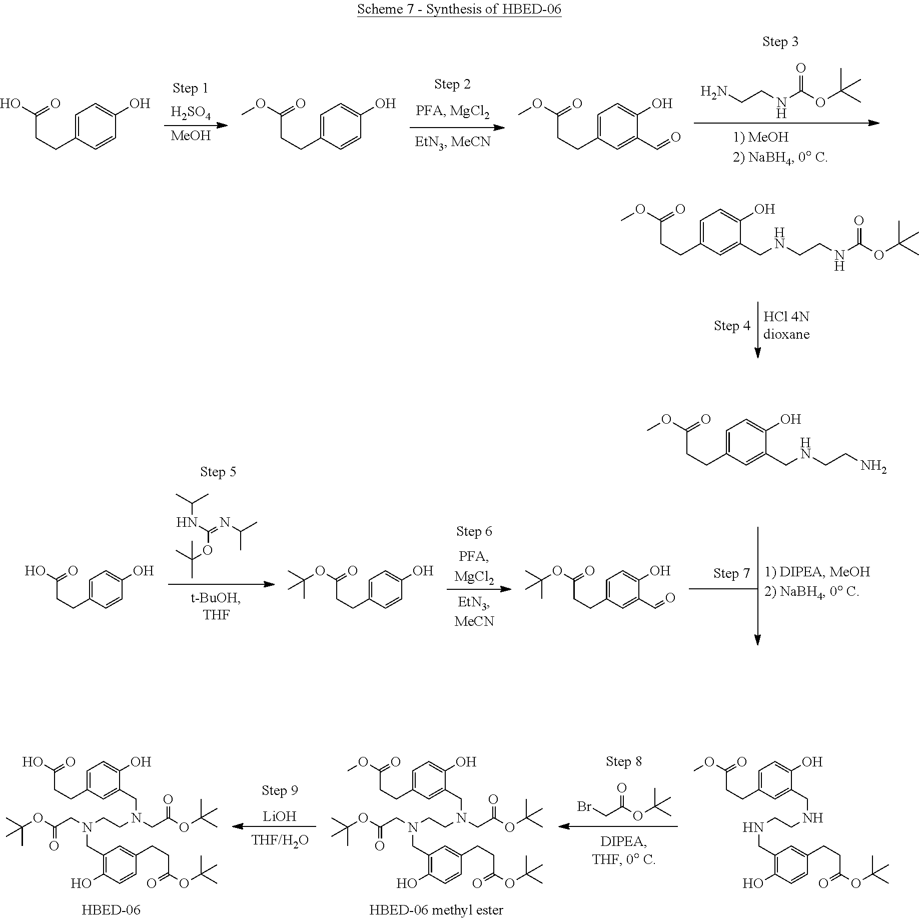

Step 1—Methyl Ester Formation

4-Hydroxyhydrocinnamic acid was dissolved in MeOH (5V). Concentrated H₂SO₄ (0.01 eq) was then added, and the reaction mixture was heated to reflux for 18 hours or until the starting material was consumed. Reaction progress was monitored by TLC. The reaction mixture was concentrated under reduced pressure and the residue was dissolved in EtOAc (2V) and washed with saturated NaHCO₃ (2V), brine (2V) and concentrated under vacuum providing the methyl ester. The product was used in the next step without further purification. ¹H NMR analysis of the obtained solid confirmed formation of the desired methyl ester product.

Step 2—Aromatic Nucleophilic Addition

To a mixture of methyl 3-(4-hydroxyphenyl)propanoate in ACN (4V), was added successively dry paraformaldehyde (8.0 eq), anhydrous magnesium dichloride (2.0 eq) and finally dry triethylamine (4.0 eq). The reaction mixture was stirred under reflux conditions for 2 hours (monitored by TLC), and then cooled to room temperature. The reaction mixture was diluted with water (10V), followed by acidification using HCl 37% until pH=3. The mixture was filtered and ACN was evaporated. The aqueous layer was extracted with DCM (8V). The organic layer was washed with brine (10V) then the solvent was removed under reduced pressure to give desired product. The product was used in the next step without further purification. ¹H NMR analysis of the obtained solid confirmed formation of the desired product.

Step 3—Reductive Amination

To a solution of methyl 3-(3-formyl-4-hydroxyphenyl)propanoate in MeOH (10 V) was added tert-butyl (2-aminoethyl)carbamate (1.2 eq). The mixture was stirred at room temperature for 4 hours. Then the solution was cooled in an ice bath. Sodium borohydride (2.2 eq.) was added in portions and then the reaction mixture was allowed to warm to room temperature then quenched with saturated NaHCO₃ (20 V). The reaction mixture was concentrated and diluted with DCM (20 V). The aqueous layer was extracted with DCM (2×10 V). The organic layer was washed with brine (10 V) then the solvent was removed under reduced pressure to give the desired product. The product was used in the next step without further purification. ¹H NMR analysis of the obtained solid confirmed formation of the desired product.

Step 4—Boc Deprotection

Tertbutyl carbamate starting material was dissolved in MeOH, and HCl 37% (5.5 eq) was added dropwise at 0° C. After being stirred at room temperature for 15 minutes (monitored by TLC), the white precipitate was filtered, washed with Et₂O (5V) and dried under vacuum to give the product as a hydrochloride salt. The product was used in the next step without further purification. ¹H NMR analysis of the obtained solid confirmed formation of the free amine product.

Step 5—tert-Butyl Ester Formation

To a solution of 4-hydroxyhydrocinnamic acid in a mixture of THF (10V) and tert-butanol (9.3V) at 0° C. was added 2-tert-butyl-1,3-diisopropylisourea (3 eq). The mixture was stirred at room temperature for 20 hours (monitored by HPLC). The reactional mixture was then filtered, and the filtrate was concentrated. DCM (6V) was added, and the organic layer was washed with saturated NaHCO₃ (3×2.4V) and concentrated under vacuum.

The solid was purified by chromatography:
Conditioning: Loading 10%
Hept/EtOAc (100:0):0.5CV
Hept/EtOAc (90:10):4.5CV
Hept/EtOAc (80:20):1.0CV

¹H NMR analysis of the obtained solid confirmed formation of the tert-butyl ester product.

Step 6—Aromatic Nucleophilic Addition

To a mixture of tert-butyl 3-(4-hydroxyphenyl)propanoate, paraformaldehyde (8.0 eq) and anhydrous magnesium dichloride (2.0 eq) in ACN (4V) was added dry triethylamine (4.0 eq). The reaction mixture was stirred under reflux conditions for 4 hours, and then cooled to room temperature. The reaction mixture was diluted with water (10V), followed by acidification using HCl 3N until pH=3. The mixture was filtered and ACN was evaporated. The aqueous layer was extracted with DCM (8V). The organic layer was washed with brine (10V) then the solvent was removed under reduced pressure to give desired product. The product was used in the next step without further purification. ¹H NMR analysis of the obtained solid confirmed formation of the desired product.

Step 7—Coupling Step Through Reductive Amination

To a solution of the free amine in MeOH (8.5V) was added DIPEA (2 eq) and the aldehyde (0.9 eq) in MeOH (1.5V). The mixture was stirred at room temperature for 5 hours, then the formed imine product was then treated with NaBH₄ (1.5 eq) at 0° C. The suspension was stirred at room temperature for 16 hours. The reaction mixture was quenched with a saturated solution of NaHCO₃ (20V) then DCM was added (20V). After filtration of the precipitate, the aqueous layer was extracted with DCM (2×10V). The combined organic layer was washed with brine (10V) and concentrated under vacuum.

The solid was purified by chromatography:
Conditioning: Loading 10%
Hept/EtOAc (70:30):10CV
Hept/EtOAc (30:70):7.5CV
Hept/EtOAc (00:100):15CV

¹H NMR analysis of the obtained solid confirmed formation of the desired product.

Step 8—Nitrogen Alkylation

To a solution of the diamine coupled product and DIPEA (2.8 eq) in THF (10V) was added ted-butyl bromoacetate (4.2 eq) at 0° C. The mixture was stirred at room temperature for 20 hours. The precipitate was filtered and the filtrate was concentrated under vacuum.

The crude product was purified by chromatography:
Conditioning: Loading 10%
Hept/EtOAc (90:10):25CV
Hept/EtOAc (85:15):10CV

¹H NMR analysis of the obtained solid confirmed formation of HBED-06 methyl ester.

Step 9—Methyl Ester Saponification

HBED-06 methyl ester was dissolved in a 1:1 THF/H₂O mixture (7-12V, typically 10V) under N₂ atmosphere in a round bottom flask. LiOH (3 eq) was added and the mixture stirred at room temperature for about 15 hours or until the starting material was consumed. Reaction progress was monitored by TLC. The mixture was then washed with diethyl ether, DIPO or other suitable organic solvent (20V). Saturated sodium chloride was added until a pH of 6-8 was reached. The aqueous phase was then extracted with ethyl acetate (3×8V) and the organic solvent concentrated under vacuum to dryness. The crude product was purified on a silica gel column (C18 fine (60M) silica: 10±1 parts by weight; gradient 18CV:1CV 100% heptane, 8CV 80:20 heptane/EtOAc, 3CV 50:50 heptane/EtOAc, 6CV 40:60 heptane/EtOAc) to provide HBED-06 as an amorphous pale yellow to colourless solid. Formation of HBED-06 was confirmed by ¹H NMR, MS and HPLC. ESI-MS: 701.

1. A method of preparing a compound of formula (V) or a salt thereof 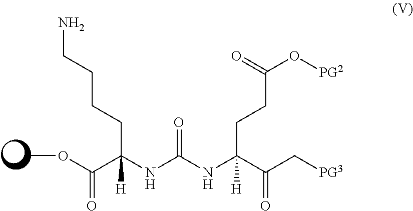
wherein [symbol]
denotes a solid support; and
PG² and PG³ are independently carboxyl protecting groups,
the method comprising reacting a compound of formula (II) with a compound of formula (III) followed by selective deprotection of PG¹, 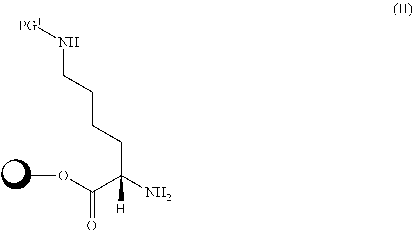
wherein: [symbol]
denotes a solid support; and
PG¹ is an amine protecting group 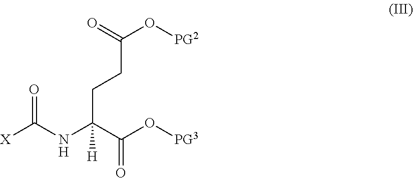
wherein:
X is a carbonyl activating group; and
PG² and PG³ are as defined for formula (V).

2. A method of preparing a compound of formula (IV) or a salt thereof 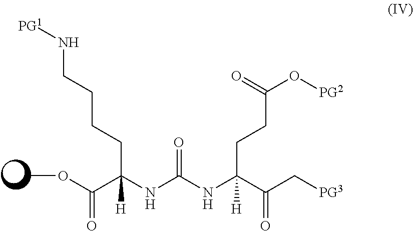
wherein [symbol]
denotes a solid support;
PG¹ is an amine protecting group; and
PG² and PG³ are independently carboxyl protecting groups;
the method comprising reacting a compound of formula (II) with a compound of formula (III), 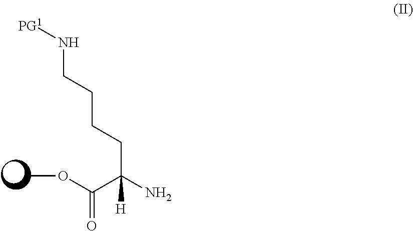
wherein: [symbol]
denotes a solid support; and
PG¹ is an amine protecting group 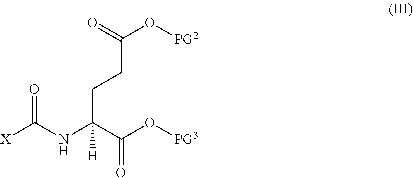
wherein:
X is a carbonyl activating group; and
PG² and PG³ are as defined for formula (IV).

3. A method of preparing a compound of formula (I) or a salt thereof: 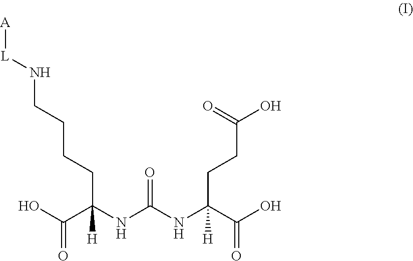
wherein
L is a covalent bond or a bifunctional linker; and
A is a ligand for a diagnostic and/or therapeutic agent;
the method comprising:
reacting the compound of formula (V) as defined in claim 1 or a salt thereof with a compound of formula (VI), formula (VII) or formula (VIII) followed by deprotection and cleavage from the solid support to provide the compound of formula (I) 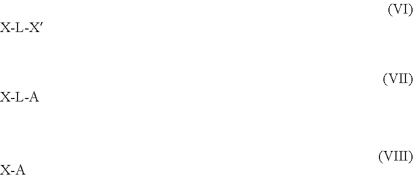
wherein
L is as defined for formula (I), with the proviso that in formula (VI), L is a bifunctional linker;
A is as defined for formula (I)
X is a group cleavable in the reaction with the compound of formula (V)
X′ is H or a group cleavable in a subsequent step reacting moiety L with a synthon of moiety A.

4. A method of preparing a compound of formula (I) as defined in claim 3, the method comprising cleaving a compound of formula (X) 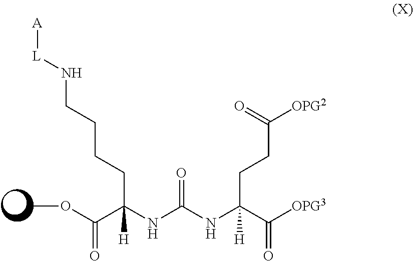
wherein [symbol]
denotes a solid support; and
L is a covalent bond or a bifunctional linker or a protected form thereof;
A is a ligand for a diagnostic and/or therapeutic agent or a protected form thereof; and
PG² and PG³ are independently H or a carboxyl protecting group,
from the solid support, and
optionally deprotecting the compound.

5. A method of preparing a compound of formula (I) as defined in claim 3, the method comprising
reacting a compound of formula (IX) 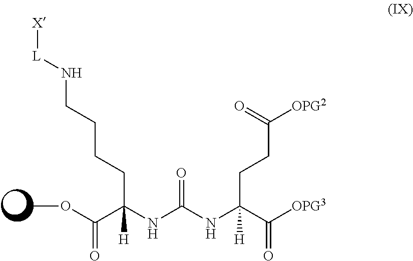
wherein
L is a bifunctional linker;
X′ is H or a group cleavable in a subsequent step reacting moiety L with a synthon of moiety A; and
PG² and PG³ are independently H or a carboxyl protecting group,
with LG_{L}-A
wherein
LG_{L} is H or a group cleavable to form a bond from moiety L to moiety A; and
A is a ligand for a therapeutic or diagnostic agent or a protected form thereof,
to provide a compound of formula (X) 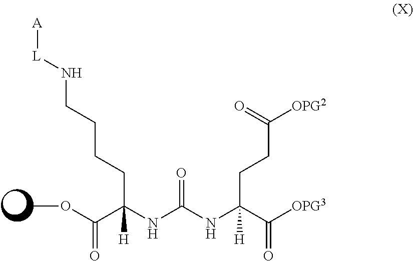
wherein [symbol]
denotes a solid support; and
L is a bifunctional linker;
A is a ligand for a diagnostic and/or therapeutic agent or a protected form thereof; and
PG² and PG³ are independently H or a carboxyl protecting group,
cleaving the compound of formula (X) from the solid support, and
optionally deprotecting the compound.

6. The method of any one of claims 1-5, wherein PG² and PG³ are the same.

7. The method of any one of claims 1-6, wherein PG² and PG³ are each tert-butyl.

8. The method of any one of claims 1-7, wherein PG¹ is alloc.

9. The method of any one of claims 1-8, wherein the solid support is a 2-chlorotrityl resin.

10. The method of any one of claims 1-9, wherein L is a multifunctional linker.

11. The method of claim 10, wherein the multifunctional linker is selected from bromoacetyl, thiols, succinimide ester, tetrafluorophenyl (TFP) ester, a maleimide, amino acids (including natural and non-natural amino acids), a nicotinamide and a nicotinamide derivative.

12. The method of claim 10 or 11, wherein the multifunctional linker is selected from:

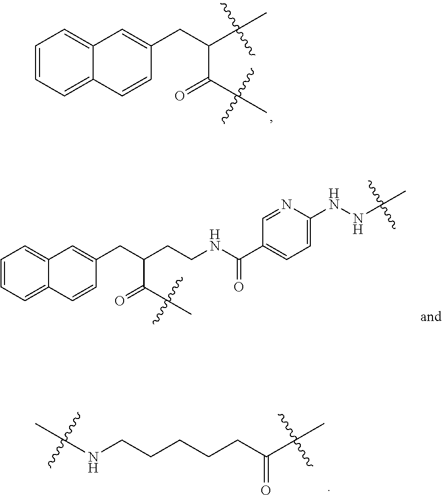 [SMILES: *C(=O)C(*)Cc1ccc2ccccc2c1 ; *NNc1ccc(C(=O)NCCC(Cc2ccc3ccccc3c2)C(*)=O)cn1 ; *NCCCCCC(*)=O]

13. The method of any one of claims 1-12, wherein A is a ligand selected from the group consisting of: TMT (6,6″-bis[N,N″,N″′-tetra(carboxymethyl)aminomethyl)-4′-(3-amino-4-methoxyphenyl)-2,2′:6′,2″-terpyridine), DOTA (1,4,7,10-tetraazacyclododecane-N,N′,N″,N″′-tetraacetic acid, also known as tetraxetan), TCMC (the tetra-primary amide of DOTA), DO3A (1,4,7,10-Tetraazacyclododecane-1,4,7-tris(acetic acid)-10-(2-thioethyl)acetamide), CB-DO2A (4,10-bis(carboxmethyl)-1,4,7,10-tetraazabicyclo[5.5.2]tetradecan), NOTA (1,4,7-triazacyclononane-triacetic acid) Diamsar (3,6,10,13,16,19-hexaazabicyclo[6.6.6]eicosane-1,8-diamine), DTPA (Pentetic acid or diethylenetriaminepentaacetic acid), CHX-A″-DTPA ([(R)-2-Amino-3-(4-isothiocyanatophenyl)propyl]-trans-(S,S)-cyclohexane-1,2-diamine-pentaacetic acid), TETA (1,4,8,11-tetraazacyclotetradecane-1,4,8,11-tetraacetic acid), Te2A (4,11-bis(carboxymethyl)-1,4,8,11-tetraazabicyclo[6.6.2]hexadecane), HBED (N,N-bis(2-Hydroxybenzyl)ethylenediamine-N,N-diacetic acid), DFO (Desferrioxamine) and analogues or derivatives thereof, such as DFO* and DFOsq (DFO-squaramide), HYNIC (6-hydrazinonicotinamide), and HOPO (3,4,3-(LI-1,2-HOPO), or a derivative thereof.

14. The method of any one of claims 1-13, wherein A is selected from DOTA, TETA, HBED, HYNIC and

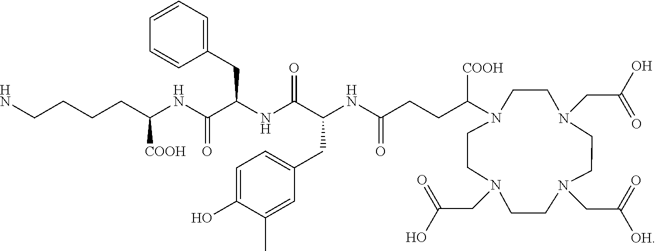

15. The method according to any one of claims 1-13, wherein LG_{L}-A selected from a compound of formula (XI) and a compound of formula (XII)

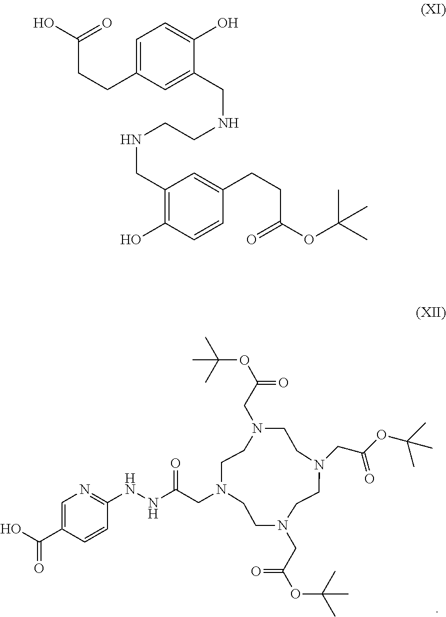 [SMILES: CC(C)(C)OC(=O)CCc1ccc(O)c(CNCCNCc2cc(CCC(=O)O)ccc2O)c1 ; CC(C)(C)OC(=O)CN1CCN(CC(=O)NNc2ccc(C(=O)O)cn2)CCN(CC(=O)OC(C)(C)C)CCN(CC(=O)OC(C)(C)C)CC1]

16. A compound or a salt thereof prepared by the method of any one of claims 1-15.

17. A compound or a salt thereof selected from any one of formulas (II)-(X): 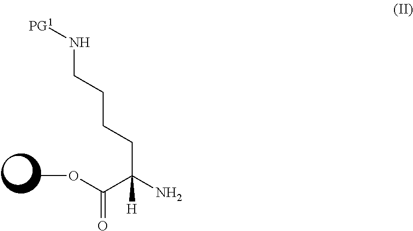
wherein: [symbol]
denotes a solid support; and
PG¹ is an amine protecting group; 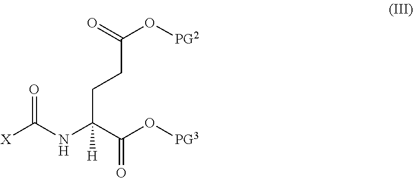
wherein:
X is —OH or a carbonyl activating group; and
PG² and PG³ are independently carboxyl protecting groups; 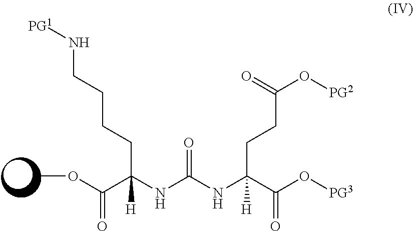
wherein [symbol]
denotes a solid support;
PG¹ is an amine protecting group; and
PG² and PG³ are independently carboxyl protecting groups; 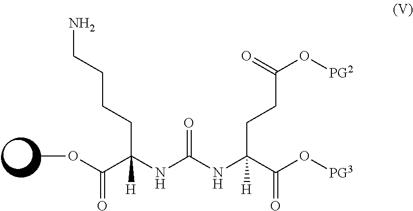
wherein [symbol]
denotes a solid support;
PG² and PG³ are independently carboxyl protecting groups; 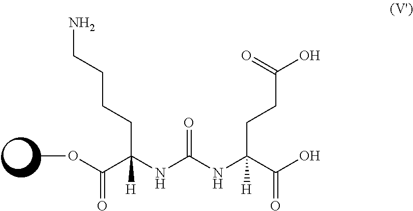 [SMILES: COC(=O)[C@H](CCCCN)NC(=O)N[C@@H](CCC(=O)O)C(=O)O]
wherein [symbol]
denotes a solid support; 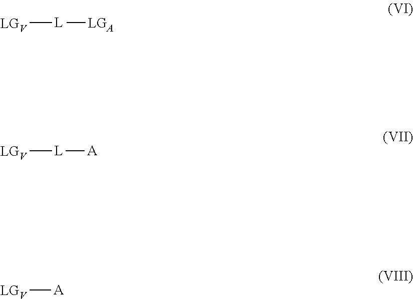
wherein
L is a bifunctional linker or a protected form of a bifunctional linker;
A is a ligand for a therapeutic and/or diagnostic agent or a protected form thereof;
LG_{V} is a group cleavable in the reaction with the compound of formula (V)
LG_{A} is H or a group cleavable in a subsequent step reacting moiety L with a synthon of moiety A; 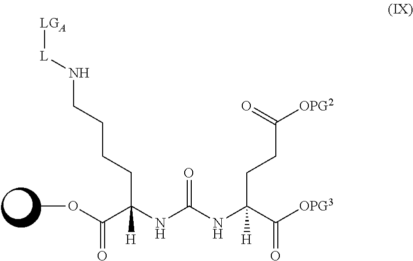
wherein L and LG_{A} are as defined in formula (VI) and PG² and PG³ are as defined for formula (V); 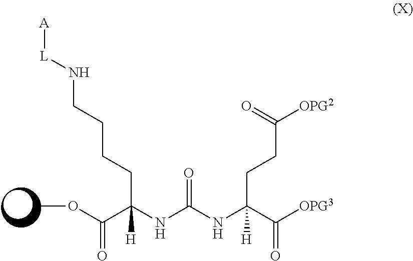
wherein [symbol]
denotes a solid support; and
L is a covalent bond or a bifunctional linker or a protected form thereof;
A is a ligand for a diagnostic and/or therapeutic agent or a protected form thereof; and
PG² and PG³ are independently H or a carboxyl protecting groups.

18. A method of preparing a compound of formula (XII) or a salt thereof 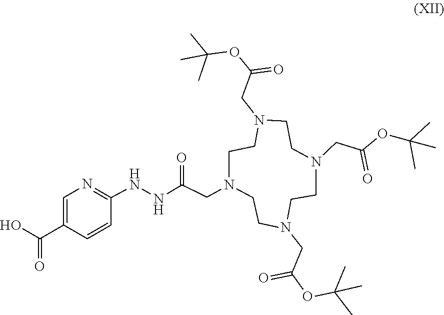 [SMILES: CC(C)(C)OC(=O)CN1CCN(CC(=O)NNc2ccc(C(=O)O)cn2)CCN(CC(=O)OC(C)(C)C)CCN(CC(=O)OC(C)(C)C)CC1]
the method comprising selectively deprotecting PG⁴ of a compound of formula (XIII) 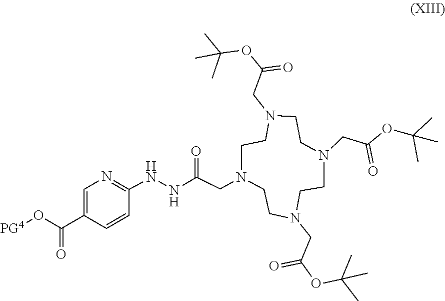
wherein
PG⁴ is a carboxyl protecting group orthogonal with a tert-butyl ester protecting group.

19. A method of claim 18, wherein the compound of formula (XIII) is prepared by reacting a compound of formula (XIV) 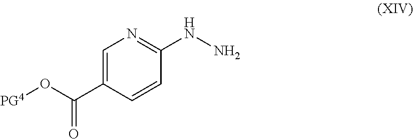
wherein
PG⁴ is a carboxyl protecting group orthogonal with a tert-butyl ester protecting group;
with a compound of formula (XV)

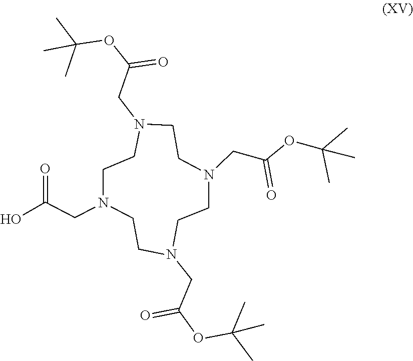 [SMILES: CC(C)(C)OC(=O)CN1CCN(CC(=O)O)CCN(CC(=O)OC(C)(C)C)CCN(CC(=O)OC(C)(C)C)CC1]

20. A method of claim 19, wherein the compound of formula (XIV) is prepared by cleaving the Boc protecting group of a compound of formula (XVI) 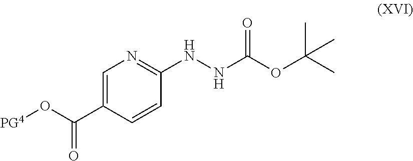
wherein
PG⁴ is a carboxyl protecting group orthogonal with a tert-butyl ester protecting group.

21. A method of any one of claims 18 to 20, wherein PG⁴ is a benzyl protecting group.

22. A compound or salt thereof prepared by the method of any one of claims 18-21.

23. A compound or a salt thereof selected from any one of formulas (XIII), (XIV) and (XVI): 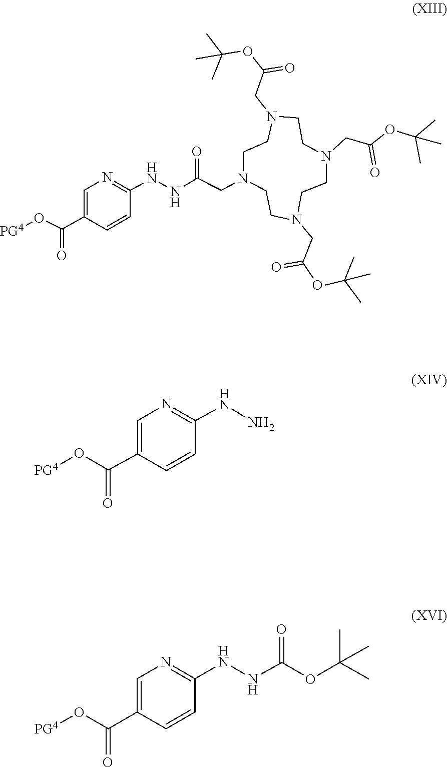
wherein
PG⁴ is a carboxyl protecting group orthogonal with a tert-butyl ester protecting group.

24. A method of claim 15, wherein the compound of formula (XII) is prepared by the method of any one of claims 18-21.

25. A method of preparing a compound of formula (IV) as defined in any one of claims 1-24 ora salt thereof, the method comprising reacting a compound of formula (II) and a compound of formula (III) as defined in any one of claims 1-24.

26. A method of preparing a compound of formula (V) as defined in any one of claims 1-24 or a salt thereof, the method comprising selective deprotection of PG¹ in the compound of formula (IV) as defined in any one of claims 1-24 to provide the compound of formula (V).

27. A method of preparing a compound of formula (V) as defined in any one of claims 1-15 ora salt thereof, the method comprising reacting a compound of formula (II) and a compound of formula (III) as defined in any one of claims 1-24 and selectively cleaving moiety PG¹.

28. The compound of claim 16 or salt thereof, for use in forming a complex with a radio isotope.

29. A pharmaceutical composition comprising the compound of claim 16 optionally complexed with a radio isotope.

30. The use of claim 28 or pharmaceutical composition of claim 29, wherein the radio isotope is selected from actinium-225 (²²⁵Ac), astatine-211 (²¹¹At), bismuth-212 and bismuth-213 (²¹²Bi, ²¹³Bi), copper-64 and copper-67 (⁶⁴Cu, ⁶⁷Cu), gallium-67 and gallium-68 (⁶⁷Ga and ⁶⁸Ga), indium-111 (¹¹¹In), iodine-123, -124, -125 or -131 (¹²³I, ¹²⁴I, ¹²⁵I, ¹³¹I) (¹²³I), lead-212 (²¹²Pb), lutetium-177 (¹⁷⁷Lu), radium-223 (²²³Ra), samarium-153 (¹⁵³Sm), scandium-44 and scandium-47 (⁴⁴Sc, ⁴⁷Sc), strontium-90 (⁹⁰Sr), technetium-99 (^{99m}Tc), yttrium-86 and yttrium-90 (⁸⁶Y, ⁹⁰Y), zirconium-89 (⁸⁹Zr).

31. A method of treating prostate cancer, comprising administering to a subject in need thereof an effective amount of a complex of a therapeutic radio isotope and the compound of claim 16 or 28 or a pharmaceutically acceptable salt thereof, or the pharmaceutical composition of claim 28.

32. Use of the compound of claim 16 or 28 or a pharmaceutically acceptable salt thereof in the manufacture of a medicament for treating prostate cancer.

33. A method of imaging a prostate cancer tumour, comprising administering to a subject in need thereof an effective amount of a complex of a diagnostic radio isotope and the compound of claim 16 or 28 or a pharmaceutically acceptable salt thereof, or the pharmaceutical composition of claim 29, and imaging the prostate cancer tumour.

34. A method of diagnosing, monitoring or prognosing a prostate cancer, comprising:
(a) administering to a subject in need thereof an effective amount of a complex of a diagnostic radio isotope and a compound of formula (I) of claim 16 or 28 or a pharmaceutically acceptable salt thereof,
(b) allowing the complex to concentrate at sites in the subject where PSMA is found; and
(c) detecting the radio isotope of the complex,
whereby detection of the radio isotope enables an image of the cancer (if present) to be created to thereby diagnose, monitor or prognose the prostate cancer.
